(19) United States
(12) Patent Application Publication
(10) Pub. No.: US 2011/0196213 A1
(43) Pub. Date: Aug. 11, 2011

(54) Display For Biological Values

(75) Inventors: Ajay Thukral, Fishers, IN (US); Stefan Weinert, Pendleton, IN (US); Abhishek Soni, Indianapolis, IN (US); Paul Galley, Cumberland, IN (US); Alan Greenburg, Indianapolis, IN (US); Ildiko Amann-Zalan, Weinheim (DE); Juergen Rasch-Menges, Schwetzingen (DE)

(73) Assignees: ROCHE DIAGNOSTICS OPERATIONS, INC., Indianapolis, IN (US); ROCHE DIAGNOSTICS GbmH, Mannheim (DE)

(21) Appl. No.: 13/017,526

(22) Filed: Jan. 31, 2011

Related U.S. Application Data
(63) Continuation-in-part of application No. 12/492,667, filed on Jun. 26, 2009.

(51) Int. Cl. A61B 5/00

(52) U.S. Cl. 600/301

(57) ABSTRACT

Methods for providing an estimated or predicted biological values in a sectioned display to assess the relative impact of a set of variables include collecting biological measurements, grouping the biological measurements based on the set of variables, evaluating the biological measurements to determine grouped estimated biological values or grouped predicted biological values, and providing the grouped estimated biological values or grouped predicted biological values within a plurality of sections in the sectioned display, wherein the plurality of sections correspond to the set of variables.

### CROSS-REFERENCE TO RELATED APPLICATIONS

This application is a continuation-in-part of U.S. patent application Ser. No. 12/492,667 filed Jun. 26, 2009 which is incorporated by reference herein.

### TECHNICAL FIELD

The disclosure relates to physiological monitoring, and in particular, to a methods and displays for providing estimated and predicted biological values from biological measurements.

### BACKGROUND

For monitoring glycemia, the American Diabetes Association (ADA) recommends the hemoglobin AlC test, hereinafter referred to HbAlC. Health care providers (HCPs) use HbAlC as a surrogate marker to evaluate a patient's glycemia over a previous 2 to 3 month period and as a target parameter by which to treat patients. For example, the HbAlC value, which can be presented as a percentage of glycated hemoglobin or in international standardized units mmol/mol (by International Federation of Clinical Chemistry, IFCC), is needed by the HCP when deciding or recommending a change to a patient's therapy. Therapy modification may include a change to, an addition to, or a switch in insulin therapy, oral medication, nutrition, physical activity, or combinations thereof in order to regulate a patient's glucose with the goal of improving a patient's HbAlC value. For a high quality determination (i.e., coefficient of variation (CV)<3%) of the HbAlC value, HbAlC assays are the norm in which blood samples are tested for the extent of glycation of hemoglobin by use of laboratory devices such as, for example, a D-10 analyzer from Bio-Rad Laboratories, or a G-7 analyzer from Tosoh Bioscience, Inc. For an approximated assessment of glycemia, HCPs alternatively use blood glucose (bG) values to determine an average glucose value, and then interpret the results to derive an estimated HbAlC value from spot monitoring blood glucose (SMBG) values. However, the estimated HbAlC value so obtained by such a method, in general, is poor in quality (i.e., CV>5%).

Other methods of solving a true mean bG value and estimating HbAlC value have been based on both the SMBG data collected during various clinical trials and the relationships derived there from. For example, many such methods use SMBG data to develop prediction models based on statistical methods. Other methods consider weighted bG value schemas with additional predicators, such as a previous HbAlC value, to determine an estimated HbAlC value using a noted study relationship. While still other methods further include transforming a bG value and then using the transformed bG value to determine the estimated HbAlC value. However, such methods have the following potential issues: model parameters typically needs returning, correlation is still generally poor (i.e., CV>5%), the standard errors are typically large, and adjustments to account for lifestyle related variations are not made such that any such reported patient specific solution is not specific enough to account for lifestyle related variations.

It is to be appreciated that one of the key limiting factors to finding a good generic algorithm which provides an accurate HbAlC estimation (i.e., determining the current HbAlC value) or prediction (i.e., determining the future HbAlC value) is the difficulty in obtaining comprehensive and detail (frequently sampled) blood glucose data under various conditions. For instance, studies having data sets based on continuous blood glucose monitoring, although providing dense data are typically conducted on relatively smaller population sizes and with durations that are relatively shorter in time than studies with SMBG data sets. With SMBG, on the other hand, there is a practical limitation of how many measurements can be collected. Since bG varies during the day, due to many factors such as physical activity, meal response, drug response (such as oral drugs or insulin) and stress and so forth, it is not possible to get an accurate picture of a glucose excursion by just a few daily measurements. This means that the SMBG data sets (i.e., time-interval based data sets) often fail to capture true bG variation of the patient with diabetes (PwD). The implication is that the resulting prediction models are normally then very study specific. Such prediction models therefore can neither be extended to account for other variables not addressed by the study(-ies) which they were based on nor used in an alternate situation to make predictions without the need for an additional clinical trial to validate such model extensions. Furthermore, as such methods fail to account for the context associated with bG measurements or in other words, to account for influence(s) of events such as carbohydrate ingestion, physical activity, insulin therapy, oral drug therapy, and so forth, such methods are generally unsuitable for determining an estimated HbAlC value of good quality (i.e., CV<3%) for a patient specific lifestyle. The lack of context associated with measurements can also limit the application of results when studying other glycemic excursion or non glycemic excursion factors (e.g., lipid profiles, insulin concentration profile, heart rate profile assuming availability of spot/continuous monitoring of the respective parameter). Finally, such methods fail to provide the estimated parameter, such as the estimated HbAlC values (or other parameters such as mean glucose, weighted glucose, fructosamine, biomarkers for various lipid levels, etc.) in a manner that can allow one to assess the relative impact of various components on a patient's overall HbAlC in a manner that would provide a quick evaluation of an implemented therapy.

### SUMMARY

In one embodiment, a method for providing an estimated or predicted biological values in a sectioned display to assess the relative impact of a set of variables is provided. The method includes collecting biological measurements, grouping the biological measurements based on the set of variables, and evaluating the biological measurements to determine grouped estimated biological values or grouped predicted biological values. The method further provides the grouped estimated biological values or grouped predicted biological values within a plurality of sections in the sectioned display, wherein the plurality of sections correspond to the set of variables.

In another embodiment, a sectioned display device for displaying grouped biological values is provided. The sectioned display device includes an input terminal for collecting both biological measurements and associated context of the biological measurements at daily times or events, memory for storing the biological measurements, the associated context of the biological measurements and instructions, and a processor in communication with the memory. The processor is operable to execute the instructions such that the instructions cause the processor to group the biological measurements based on a set of variables, evaluate the biological measurements to determine grouped estimated biological values or grouped predicted biological values, and provide the grouped estimated biological values or grouped predicted biological values within a plurality of sections in the sectioned display, wherein the plurality of sections correspond to the set of variables.

In still another embodiment, a method for selectively displaying a patient's glycated hemoglobin (HbAlC) based on various types of values is provided. The method includes collecting both bG measurements and associated context of the bG measurements at daily times or events, weighting each of the collected bG measurements based on the associated context, determining estimated HbAlC values from the weighted measurements of the collected bG measurements and determining additional types of HbAlC values. The method further includes selecting which types of HbAlC values to display and displaying the selected types of HbAlC values.

These and other advantages and features disclosed herein will be made more apparent from the description, drawings and claims that follow.

### BRIEF DESCRIPTION OF THE DRAWINGS

The following detailed description of the embodiments of the present invention can be best understood when read in conjunction with the following drawings, where like structure is indicated with like reference numerals, and in which:

FIG. 1 depicts a tabulated dataset from simulation of a Mortensen Model based system with a simplification according to the present disclosure;

FIG. 2 depicts graphically HbAlC values generated by simulation which are plotted against mean bG values;

FIG. 3 depicts graphically a disturbance model;

FIG. 4 depicts graphically results obtained from simulation that show true mean bG is linearly related to HbAlC, whereby both positive and negative glycemic variations are illustrated;

FIG. 5 depicts graphically a normal daily lifestyle pattern of an individual for a modal day that consists of an overnight period and 3 meal types: breakfast, lunch and supper;

FIG. 6 depicts in block diagram meal selections from a glycemic excursion perspective categorized per meal type, meal amount, and meal speed;

FIG. 7 depicts graphically a grouping of glucose sections by event type to characterize and quantify the sections in order to help identify parameters that provided a high correlation ratio;

FIG. 8 depicts graphically 4 weighting schemes considered in developing a prediction model for HbAlC according to the present disclosure;

FIG. 9 depicts graphically a strong linear relationship between HbAlC and post prandial bG measurement when t≧150 minutes;

FIG. 10 depicts graphically quality of estimated HbAlC for various values of a Window Center (WinCen) and a Window Size (WinSize) for lifestyle context plotted by R-squared values;

FIG. 11 depicts graphically quality of estimated HbAlC for various values of a Window Center (WinCen) and a Window Size (WinSize) for lifestyle context plotted by mean squared errors;

FIG. 12 depicts graphically a comparison between daily lifestyle weighting and no daily lifestyle weighting and showing that daily life style weighting produces lower mean squared error;

FIG. 13 depicts graphically impact of visitation period (nDays) and number of sample (nSamples) for a WinCen of 190 minutes and a WinSize of 50 minutes;

FIG. 14 depicts graphically a sampling ratio plotted against R-squared values;

FIGS. 15A-E each depict graphically a sampling schema for sampled bG data being regressed and plotted by HbAlC %, and showing that the parameters for each linear regression are in close proximity to each other;

FIGS. 16A-E each depict graphically a sampling schema for sampled bG data being regressed and plotted by HbAlC % with a prediction line (center line) and a 95% confidence interval (CI) boundaries (above and below curves) shown in the subplots;

FIG. 17 depicts in block diagram a processor based system according to one or more embodiments shown or described herein;

FIG. 18 is a flow diagram for processing data according to one or more embodiments shown or described herein;

FIG. 19 is another flow diagram for processing data according to one or more embodiments shown or described herein;

FIG. 20 is a flow diagram of a delivery method for providing grouped estimated HbAlC values in a sectioned display according to one or more embodiments shown or described herein;

FIG. 21 is an exemplary visualization of grouping biological measurements in preparation for providing grouped values in a sectioned display according to one or more embodiments shown or described herein;

FIG. 22 is a first exemplary sectioned display according to one or more embodiments shown or described herein;

FIG. 23 is a second exemplary sectioned display according to one or more embodiments shown or described herein;

FIG. 24 is a third exemplary sectioned display according to one or more embodiments shown or described herein;

FIG. 25 is a fourth exemplary sectioned display according to one or more embodiments shown or described herein;

FIG. 26 is a fifth exemplary sectioned display according to one or more embodiments shown or described herein;

FIG. 27 is a sixth exemplary sectioned display according to one or more embodiments shown or described herein;

FIG. 28 is a seventh exemplary sectioned display according to one or more embodiments shown or described herein;

FIG. 29 is an eighth exemplary sectioned display according to one or more embodiments shown or described herein;

FIG. 30 is a ninth exemplary sectioned display according to one or more embodiments shown or described herein;

FIG. 31 is an exemplary textual screen for prompting and conveying detailed information regarding the biological measurements and/or the estimated or predicted biological values according to one or more embodiments shown or described herein; and

FIG. 32 is a flow diagram of a selective display method according to one or more embodiments shown or described herein.

### DETAILED DESCRIPTION

It is to be appreciated that embodiments of the present disclosure enhance existing software and/or hardware that retrieves and processes biological measurements such as blood glucose (bG) data. The embodiments of the disclosure can be directly incorporated into existing home glucose monitors, or used for the enhancement of software that retrieves and processes biological measurements (e.g., bG data), by introducing a method for delivering estimated biological values (e.g., estimated glycated hemoglobin (HbAlC) values) of good quality from structured spot biological measurements having a coefficient of variation (CV) of less than 5% in one embodiment, and less than 3% in a preferably embodiment.

In the sections to follow, a discussion is made first to the exemplary approach used to derive the equations for providing the estimated true mean blood glucose (bG) value and the estimated glycated hemoglobin (HbAlC) value from structured spot measurements of blood glucose (i.e., bG data) collected, as per a measurement schema according to the present disclosure. It is to be appreciated that the measurement schema according to the present disclosure assumes that the PwD maintains a repeatable average behavior, whereby collection exceptions (i.e., missed testing times) are managed by the algorithm on the estimated HbAlC value. It is further to be appreciated that the utility of providing an estimated HbAlC value that demonstrates continuous blood glucose monitoring will provide a fairly accurate idea of the overall level of glycemia of the PwD, as compared to the uncertainty associated with estimating glycemia and its variation based only on spot monitoring. Additionally, certain HbAlC values have been linked to various disease states, and thus having a good estimated HbAlc value between laboratory based assays values can help identify much earlier a patient's potential risk associated to long term complications, such as micro-vascular (retinopathy, neuropathy, nephropathy) disease complications. Furthermore, providing an assessment of overall glycemia via an estimated HbAlC value of good quality can empower the PwD to manage better his/her diabetes. Alternatively, using a shorter window the algorithm can provide predicted HbAlc which allows PwD and HCP impact of current lifestyle on future HbAlc. A discussion of the methodology used to provide the estimated true mean blood glucose value, estimated glycated hemoglobin (HbAlC) value and other estimated and predicted biological values from collected biological measurements according to the present disclosure now follows.

Kinetics of Glycation of Hemoglobin

Glycation is a non-enzymatic chemical reaction wherein the glucose molecules bind with the amino acid groups of the proteins. Of the many glycated proteins hemoglobin AlC is fairly stable and one of the dominant forms of glycohemoglobin. Synthesis of HbAlC is primarily a condensation of hexose with the hemoglobin structure to form an unstable intermediate Schiff base adduct, or aldimine, followed by the Amadori rearrangement to form the stable ketoamine adduct, HbAlc. The kinetics of the glycation of hemoglobin to surrounding glucose concentration can be modeled by three differential equation, Equations (1)-(3), which are disclosed more fully by the publication of Mortensen, H. B.; “Glycated hemoglobin. Reaction and biokinetic studies. Clinical application of hemoglobin Alc in the assessment of metabolic control in children with diabetes mellitus,” Danish medical bulletin (1985), 32(6), pp. 309-328. The model according to Equations (1)-(3), is referred herein as the Mortensen Model.

Mortensen Model

$$\begin{matrix}\frac{d {H}_{bA}}{d t} = - {k}_{12} {H}_{bA} G + {k}_{21} {H}_{bA 1 d} , & ( 1 ) \\ \frac{d {H}_{bA 1 d}}{d t} = {k}_{12} {H}_{bA} G - ( {k}_{21} + {k}_{23} ) {H}_{bA 1 d} + {k}_{32} {H}_{bA 1 C} , and & ( 2 ) \\ \frac{d {H}_{bA 1 c}}{d t} = {k}_{23} {H}_{bA 1 d} - {k}_{32} {H}_{bA 1 C} . & ( 3 )\end{matrix}$$

In the Mortensen model, the term H_{bA} represents the sub-pool of erythrocytes of same age, whereby the pool consists of cohorts of erythrocytes of varying age. The behavior of each cohort is represented by a corresponding set of Equations (1)-(3). From the Mortensen publication, the k parameters used in the model are known as follows: k₁₂=5.76 mmol/l/min; k₂₁=0.006/min; k₂₃=0.000852/min; and k₃₂=0.000102/min. Next, to test the utility of the Mortensen model in helping to generate a basic relation between HbAlC and bG, glycation simulations were run on simulated data for meal related bG excursions which is discussed hereafter.

Glycation Simulation Setup

Meal related bG excursions were evaluated first using simple mathematical formulas, whereby simulated data helped to generate a basic relation between HbAlC and bG. It is to be appreciated that the glycation of erythrocytes is a continuous process. However, the erythrocytes have finite lifespan of approximately 120 days. Depending on problem needs one can use other lifespan values such as ranging more or less from 90˜120 days to cover different population groups and/or physiological conditions. This means that in addition to the glycation, erythrocytes are continuously being added and removed from the glycation process. As the aged erythrocytes are replaced, the state of glycation of all the cells has to be managed. From a simulation perspective, instead of using Equations (1)-(3) for each cell, a simplification was done by grouping cells into cohorts of equally aged cells. In particular for the glycation simulation setup, n numbers of cohorts of erythrocytes were considered, whereby each of the cohorts was described by the set of the 3 differential equations (Equations (1)-(3)). Each cohort is assumed to have a life span of n days. When a cohort's maximum age is reached, a new cohort replaces it. The simulation handled this by resetting the 3-states of the oldest cohort (i.e. when the age of the cohort reaches its life-duration of 120 days) to the state of a fresh cohort of erythrocytes with non-glycated hemoglobin. In all, there were n sets of differential equations used in the simulation, whereby each set of equations represented a state of the corresponding cohort.

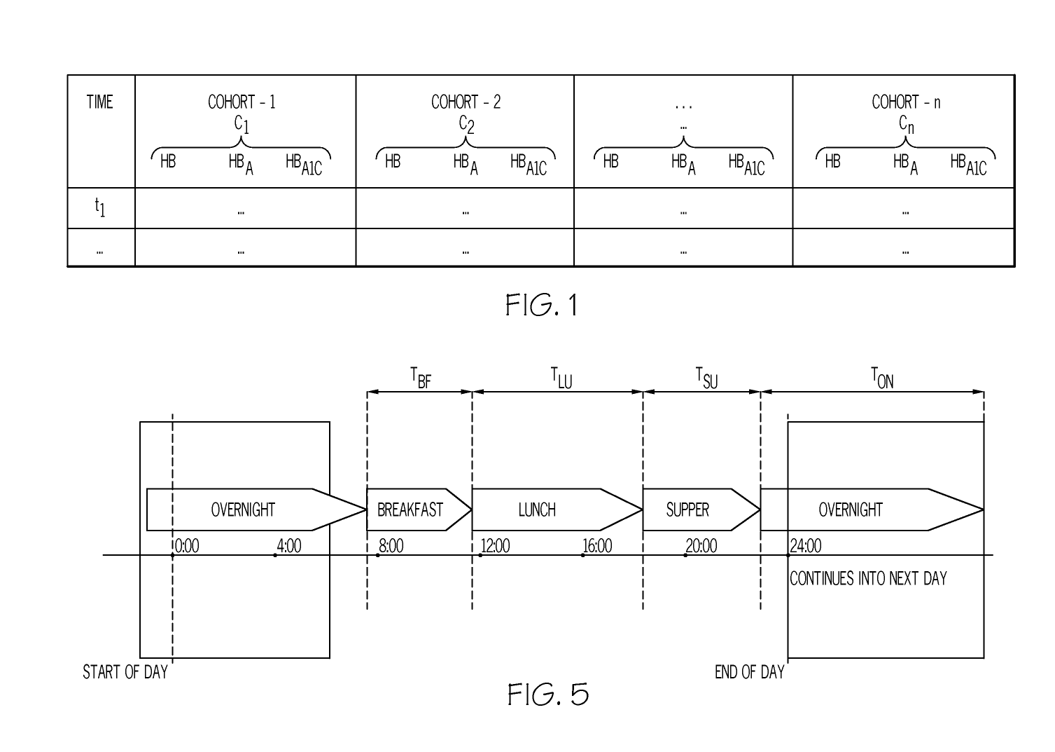

The 3n states were stored in columns as shown schematically in FIG. 1, which represent a tabulated dataset from the simulation of the Mortensen Model system using the above mentioned simplification. At each new time instant, the values for each of the states were recorded in the next new row as a record set, whereby glycation of each cohort at any given time is the value of the 3^{rd} state. The net HbAlC in percentage (%) can be given by summing the HbAlC state for each of the cohort according to Equation (4):

$${HB}_{A 1 C} = 100 {\sum }_{i = 3 , 9 , …}^{n} {x}_{i} , \qquad ( 4 )$$

where the summation counter i is the column corresponding to the Hb_{Alc} state. Using Equations (1)-(4), HbAlC can be simulated for an arbitrary bG profile. The true mean blood glucose value, b̅G̅ can be thus given by Equation 5:

$$\overline{bG} = \frac{AUC}{Duration} , \qquad ( 5 )$$

where AUC is the area under a continuous bG excursion curve. However, when bG measurements are sparse and non-continuous, as in the case of spot bG measurements, then it is to be appreciated that Equation 5 is no longer valid. Accordingly, a new relationship was derived in order to estimate the true mean bG as follows.

Simulated Cases

Under the above mentioned idealized setup, the relationship between periodic glucose profiles and corresponding HbAlC values was then examined for deriving useful insights and relationships. Specifically, two profiles were examined: (1) Sinusoidal glucose profile with offset (Equation (6)); and (2) Gamma function profile with offset (Equation (8)). These functions can be seen as representative of the meal event with post-prandial glucose behavior for varying levels of control, whereby constant glucose is a special case of both of the functions. For the sinusoidal glucose profile, Equation (6) is defined as:

$$bG = {bG}_{const} + \frac{A}{2} ( 1 - cos ( \frac{2 π}{T} t ) ) , \qquad ( 6 )$$

where, bG_{const} provides the steady state offset and

$$\frac{A}{2} ( 1 - cos ( \frac{2 π}{T} t ) )$$

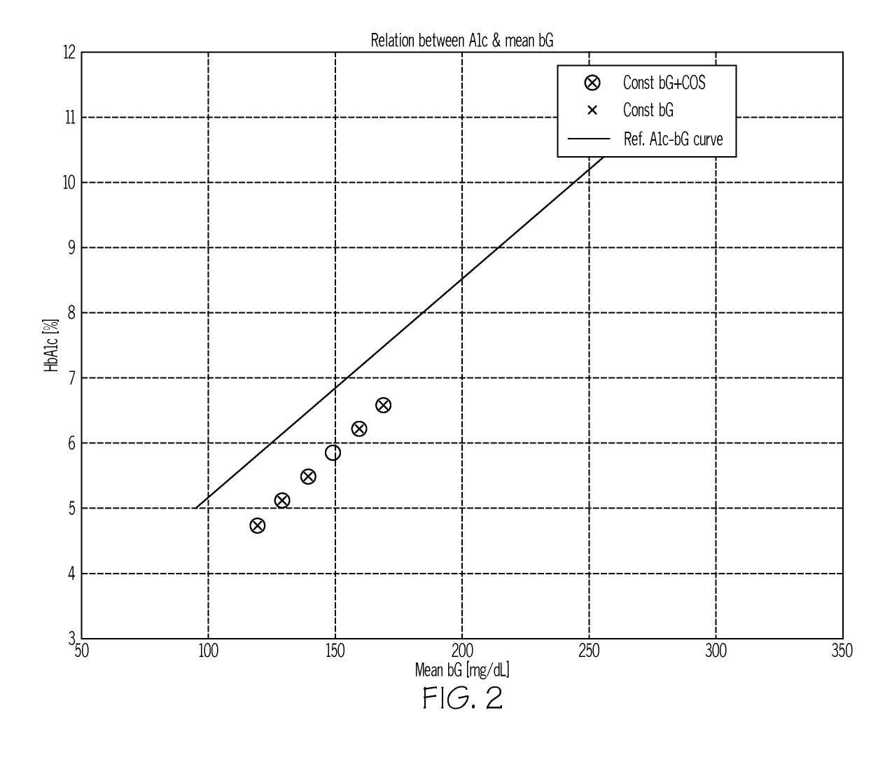

is the cosine curve with amplitude A and period T. FIG. 2 shows the HbAlC values generated by the simulation plotted against the mean bG values. The results in FIG. 2 show that the true mean bG for the continuous glucose profile and simulated HbAlC is approximately linear as shown by the ‘o’ symbols. It also shows that HbAlC obtained by a constant bG and that from oscillating bG are approximately identical if they have the same mean bG value. If comparing the HbAlC resulting from two sinusoidal inputs, which are identical except for the frequency, the HbAlC from the slower varying signal will have comparatively higher glycation rate. The rate of signal has an effect but under conditions of interest it is small. The solid curve shows a known relationship between HbAlC and mean bG and is used as a reference.

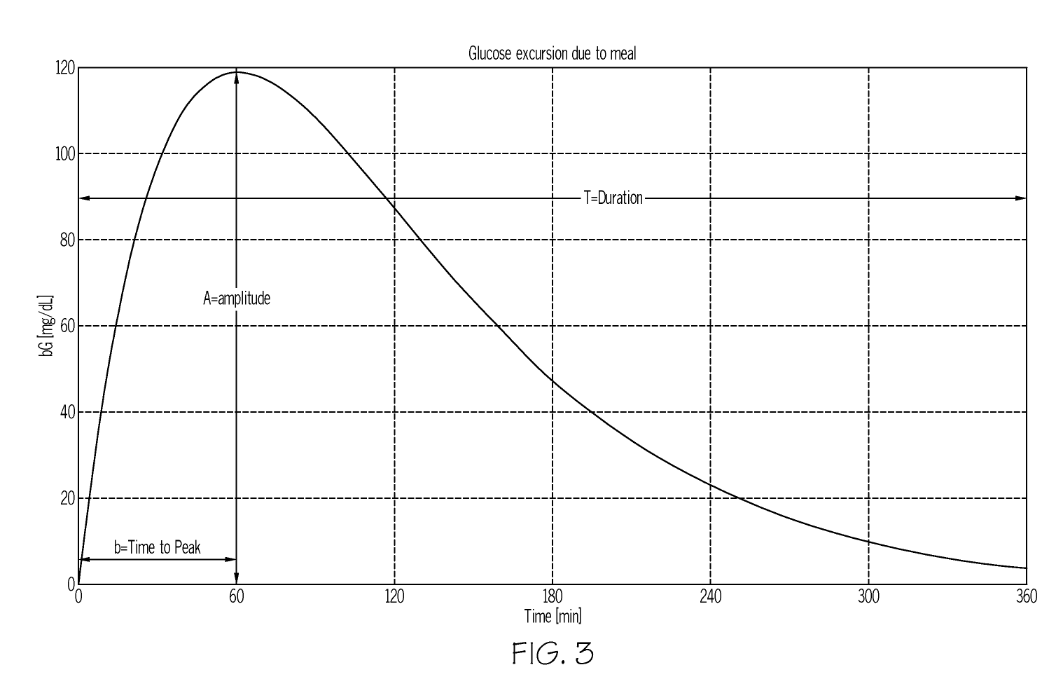

The disturbance model (in which the gamma function was used to model disturbance) is shown by FIG. 3, and is described by the function defined by Equation (7):

$$f ( t ) = \frac{{t}^{α - 1} {e}^{- t / β}}{{β}^{a} Γ ( α )} , t \geq 0. \qquad ( 7 )$$

In this simulation embodiment, the Gamma function was used to represent the post meal glucose excursion. Grossly approximated, the model shows a two (2) compartment model of glucose in a post prandial state. It has been used primarily to understand the impact of glucose varying from the aspects of different rates of postprandial rise, decay and magnitude of an excursion. The parameters α and β approximately represent the number of compartments and time to peak. In fact, if α is set to 2, a 2^{nd} order compartment model is considered with a time constant for both compartments equal to β (2^{nd} order system with repeated poles). Therefore, the above function according to Equation (7) simplifies to:

$$f ( t ) = \frac{t {e}^{- t / β}}{{β}^{2}} , t \geq 0.$$

The peak value of the function ƒ(t) is reached when t=β. The peak value is then

$$\frac{1}{eβ} .$$

Therefore, the glucose excursion used herein can be defined by Equation 8:

$$bG ( t ) = {bG}_{const} + \frac{A e}{β} t {e}^{- t / β} , t \geq 0 , \qquad ( 8 )$$

where A is the peak bG value with respect to bG_{const}.

The response of HbAlC to glucose excursions for various time to peak and peak values was then studied both analytically and in simulation. The Gamma functions for the various combinations of the parameters studied are listed in Table 1.

TABLE 1
Parameter settings for Gamma function

| Parameter | Values |
|---|---|
| Constant, bG_{const} [mg/dL] | 80, 100, 120, 140 |
| Amplitude, A [mg/dL] | Positive (+ve) glucose push |
| | 0, 40, 60, 80, 100, 120, 140, 180, 300 |
| | Negative (−ve) glucose push |
| | 0, 40, 60, 80 |
| Periodicity [hour] | 4, 6, 8, 12, 24 |
| Time to peak, β [minute] | 30, 60, 90, 120 |

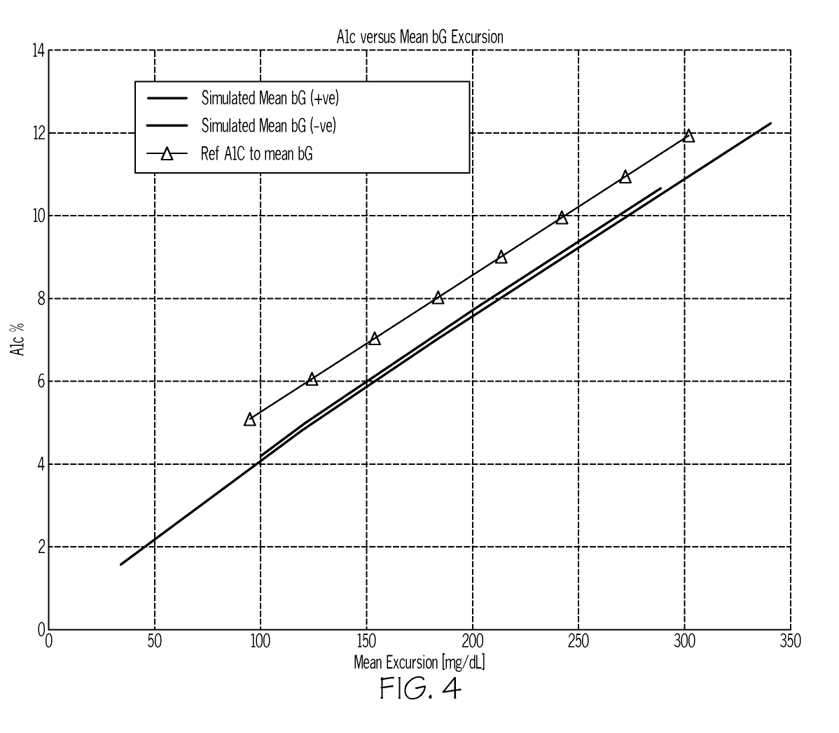

The results obtained from simulation show that the true mean bG is linearly related to HbAlC. This linear relationship is shown by FIG. 4, whereby both positive and negative glycemic variations are illustrated. The solid line above 4% HbAlC is for positive glycemic variation with respect to a constant 100 mg/dL signal and the dashed line below 4% HbAlC is for the negative glycemic variation. In order to provide a more accurate (and thus more useful) estimated HbAlC value e.g., having less than 3% CV, a more accurate estimated true mean bG is needed. However, it is to be appreciated that in the case of spot measurement devices, increasing the data set of bG measurements upon which to base a more accurate estimated true mean bG is not practical as a PwD can only tolerably comply with about 3 to 6 measurements daily. Additionally, bG values are used in intensive insulin therapy to primarily regulate glucose to target. This means bG measurement timings are dependent on the requirements of intensive therapy and not on providing a better estimate of true mean bG. This is especially true in the case of Type I diabetic patients. Furthermore, for practical considerations the bG measurement cannot be limited to a specific time instant. And finally, the data analyzed normally covers two consecutive patient visits to the HCP. The period between visitations can range from 3 to 4 months. Accordingly, with the above issues in mind, specifying a time window for bG measurement was determined by the inventors to be more realistic. These issues were examined analytically using a gamma function profile, which is discussed hereafter in later sections. An example of a normal lifestyle of a PwD is now provided in order to illustrate the lifestyle aspects and context-based measurements that are collected according to a measurement schema of the present disclosure.

Lifestyle Aspects and Context Based Measurements

Intensive therapy addresses the occurrences of bG excursion and provides insulin dosing rules for correcting events such as meals, exercise, medication, etc. This leads to the term “lifestyle” which captures the properties/characteristics of the occurrences of meal events, exercise events, medication events, etc., for a PwD. Lifestyle thus has a strong connotation of daily habits. In the following example, the habits are limited to meals, but other embodiments may be extended to include other events captured, for example, physical activity, intake of oral drugs, and other daily activities.

In the following example, one daily lifestyle pattern (habit) examined consisted of an overnight period and a day period consisting of multiple meals and snacks for a patient. The daily lifestyle pattern repeats itself over a period of months whereby the timing of meals varied randomly around expected meal times. The size and composition of the meal was similarly modeled by assigning the parameters of the gamma function values generated from statistical distribution. In general, it was assumed that by considering more or less a 3 month time frame, the persistent average behavior would be observed in HbAlC value even though from meal to meal there could be potentially large variability. Thus, in the given example, a modal day consisted of an overnight period and 3 meal types: breakfast, lunch and supper as is shown in FIG. 5, which is an example of a normal lifestyle for an individual. It is to be appreciated that snacks are ignored in this illustrated embodiment for simplicity but such can be introduced in other embodiments without impacting the general approach. From either questionnaire or systematic data collection, time periods covering these events are collected.

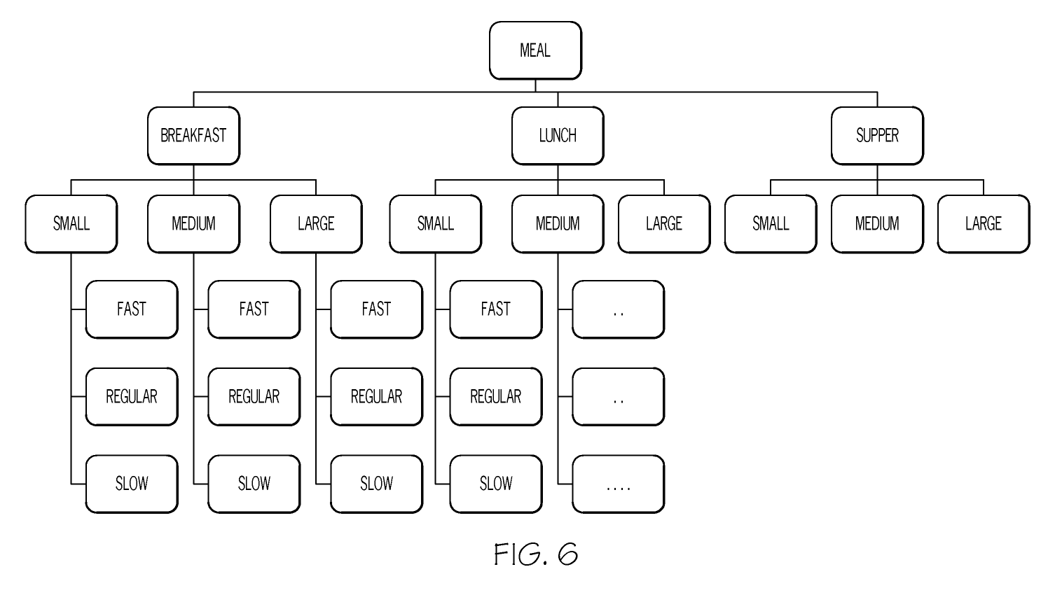

Further complexity in glucose excursion characteristics is addressed by modeling a range of meal content characterized generally by amount and speed. Meal content is given by meal composition and amount of meal which relates to speed and duration of glucose absorption. It is observed that individuals have repeatability in their meal selection, which from a glycemic excursion perspective can be classified by its speed and amount. In one embodiment, as shown in FIG. 6, meals are categorized per meal type, meal amount, and meal speed. Similarly, in other embodiments, additional meals (or less meals if such more accurately reflects a patient's eating habits), exercise (physical activity), stress, alternate states, and medication can be characterized and modeled. For example, an alternate state category may be used to capture the change in physiological metabolic state, such as brought on by stress, a menstrual cycle, exercise, or medication which leads to change in insulin resistance, insulin sensitivity, glucose utilization, and so forth.

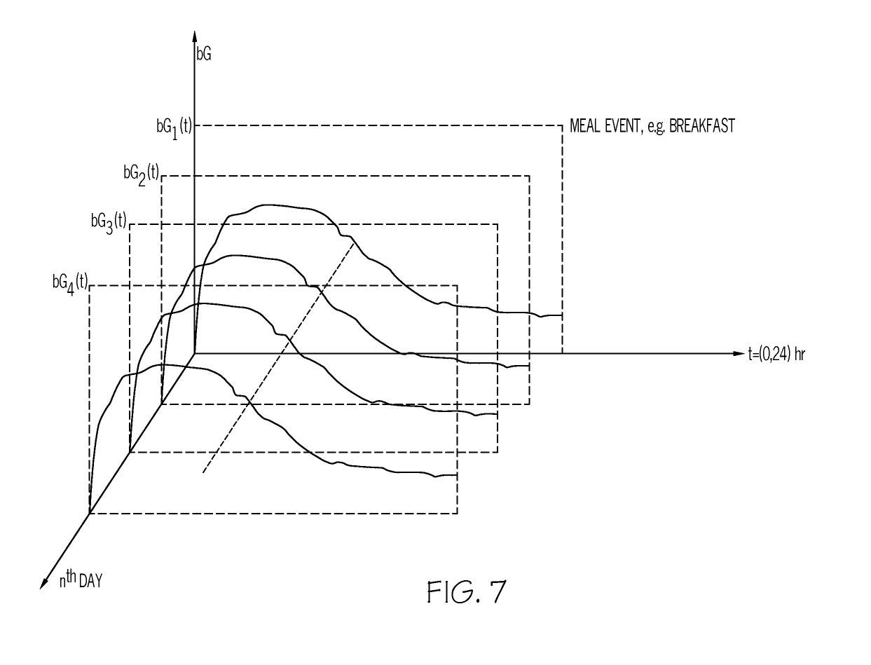

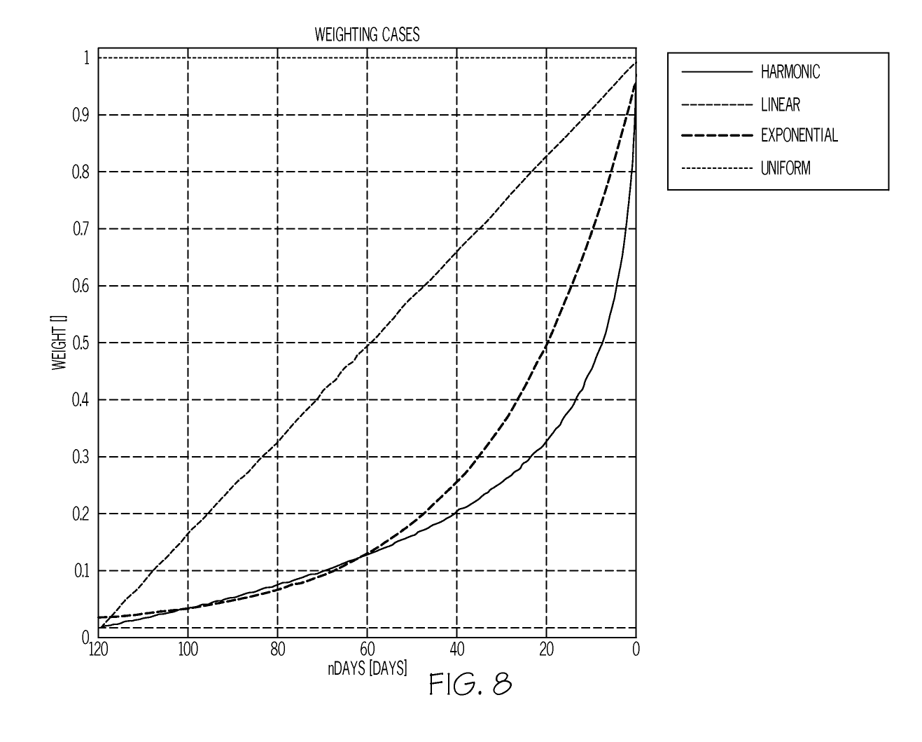

Mathematically, then, statistical properties were assigned to each of the categories. As per the above description, meals were further classified by 3 broad categories of meal speed: fast, regular and slow, and meal amount is similarly classified into 3 categories as: small, medium and large. Other terms used were less than normal, normal and more than normal. While specific categories are presented herein, it should be appreciated that other additional or alternative categories may be used to provide a generalization of a different problem. The latter part described better the majority of the cases. For purposes of simplification of the simulation, physical activity was assumed to be fixed. Thus, grouping glucose sections by event type, for instance meals, and further sub-grouping them by characterizing the meal size and speed, allows one to characterize and quantify them, which is illustrated by FIG. 7. Such context based grouping and then examining the average behavior helped to identify parameters that provided a high correlation ratio. Using the normal lifestyle described above, along with the nine meal categories, a fairly wide range of post prandial behavior for an individual is covered. The gamma function according to Equation 8 was then used in the analytical analysis as well as in simulation to study the impact of lifestyle in the derivation of the relation between HbAlC and bG measurements. It is to be appreciated that how the lifestyle pattern is sectioned and correlated can be varied based upon each patient observed habits and by using other distribution methods in other embodiments.

It is to be appreciated that in reality the glucose profiles of a PwD are richer in their response and are potentially harder to characterize. The richness is associated with multiple physiological factors influencing the overall glucose state. However, assuming that the meal related glucose push is dominant, the PwD is working towards regulating glucose to a target value by means of medications, diet control and exercise and their combinations Inherently there is an objective of achieving euglycemia at all times. By averaging many such responses, however, the glucose effect on glycemia can be estimated based on the relations derived using the gamma function. The arbitrary meal response curves shown in FIG. 7 are thus represented by the gamma function (Equation 8), which was then used to derive a relation for true mean bG and peak amplitude A. Additionally, the following provides a theoretical basis for the new lifestyle based approach.

Mean Value for Gamma Function

It is to be appreciated that the gamma function ƒ(t) is neither symmetric nor periodic. We define parameter T which is the time duration between consecutive meal. Considering the exponential properties, the decay of a pure exponential curve to 99% of its starting value is equal to 4 times the time constant. Therefore, the gamma function according to Equation 8 is basically a 2^{nd} order differential equation with repeated poles. The time constant for the gamma function is thus 1/β, and the mean value can be determined by considering the waning factor n, which is defined as:

$$n = \frac{T}{β} or T = n * β ,$$

where n=3, 4.

The mean bG value for

$$\frac{A e}{β} t {e}^{- t / β}$$

(from Equation 8) is now derived. If we consider,

$$g ( t ) = \frac{A e}{β} \frac{1}{T} {\int }_{0}^{T} t {e}^{- t / β} d t ,$$

and integrate g(t) by parts, the following Equations (9)-(13) are provided:

$$\begin{matrix}g ( t ) = \frac{A e}{β} ( {\frac{1}{T} [ t \frac{{e}^{- t / β}}{- 1 / β} ]}_{0}^{T} - \frac{1}{T} {\int }_{0}^{T} ( 1 ) \frac{{e}^{- t / β}}{- 1 / β} d t ) , & ( 9 ) \\ g ( t ) = \frac{A e}{β} ( {\frac{1}{T} [ t \frac{{e}^{- t / β}}{- 1 / β} ]}_{0}^{T} - {\frac{1}{T} [ \frac{{e}^{- t / β}}{( - 1 / β ) ( - 1 / β )} ]}_{0}^{T} ) , & ( 10 ) \\ g ( t ) = \frac{A e}{β} ( {\frac{1}{T} [ t \frac{{e}^{- t / β}}{- 1 / β} ]}_{0}^{T} - {\frac{1}{T} [ {β}^{2} {e}^{- t / β} ]}_{0}^{T} ) , & ( 11 ) \\ g ( t ) = \frac{A e}{β} ( - {\frac{1}{T} [ t {βe}^{- t / β} + {β}^{2} {e}^{- t / β} ]}_{0}^{T} ) , & ( 12 ) \\ g ( t ) = \frac{A e}{β} ( - \frac{1}{n β} [ n {ββe}^{- n β / β} + {β}^{2} {e}^{- n β / β} ] + \frac{1}{n β} [ 0 + {β}^{2} {e}^{- 0 / β} ] ) , & ( 13 )\end{matrix}$$

where T=nβ. Note, however, the mean value is a function of β, but if T is expressed in terms of β, then β falls out, which further simplifies to

$$g ( t ) = \frac{A e}{β} ( \frac{1}{n} β ( 1 - ( n + 1 ) {e}^{- n} ) ) ,$$

and finally,

$$\overline{bG} = \frac{A e}{n} ( 1 - ( n + 1 ) {e}^{- n} ) .$$

In this manner, when the waning factor n equals 3, the mean value b̅G̅ is 0.726A, and when the waning factor n equals 4, the mean value b̅G̅ is 0.617A. Thus, the mean value b̅G̅ is a function of amplitude A and the waning factor n. Adding the basal glucose level term bG_{const}, the mean value b̅G̅ can be then defined by Equation (14) as:

$$\overline{bG} = \frac{Ae}{n} ( 1 - ( n + 1 ) {e}^{- n} ) + {bG}_{Const} . \qquad ( 14 )$$

Thus, if a peak bG value is measured, then the true mean value b̅G̅ could be estimated since the waning factor n given the lifestyle can be determined by

$$n = \frac{DurationBetweenMeal , T}{TimeToPeak , β} .$$

So, for a given gamma function one could simply state that for the mean value, b̅G̅=kA+ b̅G̅_{Const}. Based on simulation of the Mortensen model, as shown by FIG. 2, it is noted that HbAlC is linearly related to true mean bG. Thus, HbACl may be defined by Equation (15) as:

HbAlC=K b̅G̅+constant (15).

From the above derivation, it is also clear that both meal size and meal duration (associated with speed) influences the degree of glycation. Next, a discussion of the process used to characterize a PwD's lifestyle is provided. Equation (15) is central to derivations presented in latter paragraphs. It is to be appreciated that the relationship between estimated mean bG and the parameters are dependent on context and sampling assumptions. Accordingly the parameters in equation (15) (K, constant) can have potentially different values in other situations. Another example to which the above approach can be applied is the estimation of fructosamine based of the biological measurement blood glucose.

Lifestyle (Meal Only)

As discussed above, the day, as per the lifestyle, is divided into appropriate sections where the bG traces for each day are sectioned and each like sections grouped (e.g., FIG. 7). The bG data covering number of days are grouped into sections as mentioned in the above embodiment comprise: a fasting section, a breakfast section, a lunch section, and a supper section. Starting from continuously sampled data, the mean value b̅G̅ is approximately given by Equation (16) as:

$$\overline{bG} = \frac{{\sum }_{i = 1}^{n} {bG}_{i}}{n} . \qquad ( 16 )$$

For a meal related section, the gamma function is described by the peak value A with respect to the basal or fasting bG and time to peak, β for bG. The parameters are summarized in Table 2.

TABLE 2
Meal characteristics

| | Fast | Regular | Slow |
|---|---|---|---|
| Small | A_{S}^{BF}, β_{Fast}^{BF} | A_{S}^{LU}, β_{Regular}^{LU} | A_{S}^{SU}, β_{Slow}^{SU} |
| Medium | A_{M}^{BF}, β_{Fast}^{BF} | A_{M}^{LU}, β_{Regular}^{LU} | A_{M}^{SU}, β_{Slow}^{SU} |
| Large | A_{L}^{BF}, β_{Fast}^{BF} | A_{L}^{LU}, β_{Regular}^{LU} | A_{L}^{SU}, β_{Slow}^{SU} |

In terms of analysis then, the meals are then characterized to cover a time period, such as for example, a 2-4 month period between HCP visitations, in the following manner. For breakfast type meals, the total number of breakfasts is represented by the term m^{BF}, and the ratio of the number of small breakfast meals, medium breakfast meals, large breakfast meals and no breakfast meals are represented by α_{SMALL}^{BF}, α_{MED}^{BF}, α_{LARGE}^{BF} and α_{Φ}^{BF}, respectively. Total breakfasts m^{BF} can then be defined according to Equation (17) as:

α_{SMALL}^{BF}m^{BF}+α_{MED}^{BF}m^{BF}+α_{LARGE}^{BF}+α_{Φ}^{BF}m^{BF}=m^{BF} (17).

Similarly, meal speeds for fast, regular, and slow meals are represented by the terms: λ_{FAST}^{BF}, λ_{REG}^{BF}, and λ_{SLOW}^{BF}, respectively. Therefore, total breakfasts m^{BF} can also be defined according to Equation (18) as:

λ_{FAST}^{BF}m^{BF}+λ_{REG}^{BF}m^{BF}+λ_{SLOW}m^{BF}+α_{Φ}^{BF}m^{BF}=m^{BF} (18).

It is assumed that on average for each meal amount category there is a breakdown for meal speed with the same ratios. In other words, for example, small breakfast meals m_{SMALL}^{BF} can be defined according to Equation (19) as:

λ_{FAST}^{BF}α_{SMALL}^{BF}m^{BF}+λ_{REG}^{BF}α_{SMALL}^{BF}m^{BF}+λ_{SLOW}^{BF}α_{SMALL}^{BF}m^{BF}=m^{BF} (19).

Equation (16) the bGᵢ terms on the right hand side are grouped as per FIG. 5 to derive a simplified mean glucose relationship using relationship b̅G̅=kA+bG_{Const} for a Gamma function of amplitude A (derived earlier). The FIG. 5 in this example consists of overnight and 3 meal sections breakfast, lunch and supper. The overnight part of the day in this example is generally the sleep period. During this period, the physical activity is minimal. Meal affects are waning out, insulin bolus affects are also petering out. There are other effects such as, for example, the dawn phenomenon caused by growth hormones, which are especially dominant in adolescents. Another example covers medication, wherein the effect of medication on the glucose dynamic response on the drugs respective pharmacokinetics and pharmacodynamics is determined. However, it is anticipated that during the overnight period, the overnight mean blood glucose value, represented by the term b̅G̅_{ON}, is converging to a desired target. Accordingly, b̅G̅_{ON} is the mean bG obtained by considering all bG values covering a fasting section, and covering all the overnight sections. The mean bG component for the overnight section is then given by Equation (20) as:

$${\overline{bG}}_{1} = \frac{{T}_{ON}}{24} {\overline{bG}}_{ON} , \qquad ( 20 )$$

where T_{ON} covers time duration for overnight part as illustrated in FIG. 5.

What can constitute fasting bG values requires more specifics. For example, pre-meal bG measurements could be grouped as fasting bG values under certain conditions, overnight bG measurements, early morning bG measurements. Average of such measurements approximately represents the mean bG for the overnight period. Then the component required for b̅G̅_{ON} is given by Equation (21) as:

b̅G̅_{ON}= b̅G̅_{Fasting} (21).

Next, given the first predictor term b̅G̅_{Fasting}, which covers the overnight period, the remaining are the meals related excursion with respect to b̅G̅_{Fasting}. So each of the meal which are gamma function thus can be defined according to Equation (22) as:

b̅G̅=KA+ b̅G̅_{FASTING} (22),

where A is the peak disturbance with respect to b̅G̅_{Fasting}.

Determination of “A” for the case when various glucose excursions due to different meals is now explained. As explained earlier and summarized by FIG. 5 and FIG. 6 the excursions are due to the 3 normally eaten meals and then each meal characterized by its size and speed. For illustration purpose, consider the breakfast part first. Next, if consider small breakfast meals and include all meal speeds, then the area under the gamma function according to Equation (23) as:

$$( {α}_{SMALL}^{BF} {m}^{BF} ) {T}^{BF} {\overline{bG}}_{SM}^{BF} - {\overline{bG}}_{FASTING} = {T}^{BF} {\sum }_{i = 1}^{{α}_{SMALL}^{BF} {m}^{BF}} {K}_{i}^{BF} {A}_{SMALL , i}^{BF} , \qquad ( 23 )$$

which covers all small breakfast meals. The term T^{BF} is the time duration between start of breakfast to start of lunch. Similar equations can be written for medium and large breakfasts, which when combined results in Equation (24), which is defined as:

$${m}^{BF} {T}^{BF} \overline{bG} - {\overline{bG}}_{FASTING} = \underbrace{{T}^{BF} {\sum }_{i = 1}^{{α}_{SMALL}^{BF} {m}^{BF}} {K}_{i}^{BF} {A}_{SMALL , i}^{BF}}_{Term - 1} + \underbrace{{T}^{BF} {\sum }_{i = 1}^{{α}_{MED}^{BF} {m}^{BF}} {K}_{i}^{BF} {A}_{MED , i}^{BF}}_{Term - 2} + … \underbrace{{T}^{BF} {\sum }_{i = 1}^{{α}_{LARGE}^{BF} {m}^{BF}} {K}_{i}^{BF} {A}_{LARGE , i}^{BF}}_{Term - 3} + \underbrace{{T}^{BF} {\sum }_{i = 1}^{{α}_{φ}^{BF} {m}^{BF}} {K}^{BF} {A}_{φ}^{BF}}_{Term - 4} . \qquad ( 24 )$$

The term A_{φ}^{BF} is of course zero. The number of meals considered in the equation covers a time window of interest. Such a window may range from 2 months to 4 months, or may be as few as 7 day to 30 days, if an estimated prediction is desired as explained in a later section.

If Term-1 is considered, then the term Kᵢ^{BF}, meal speed, can now be factored out as a constant. The result is shown by Equation (25).

$${\sum }_{i = 1}^{{α}_{SMALL}^{BF} {m}^{BF}} {K}_{i}^{BF} {A}_{SMALL , i}^{BF} = {K}_{FAST}^{BF} {\sum }_{i = 1}^{{λ}_{FAST}^{BF} {α}_{SMALL}^{BF} {m}^{BF}} {A}_{SMALL , i}^{BF} + {K}_{REG}^{BF} {\sum }_{i = 1}^{{λ}_{REG}^{BF} {α}_{SMALL}^{BF} {m}^{BF}} {A}_{SMALL , i}^{BF} + {K}_{SLOW}^{BF} {\sum }_{i = 1}^{{λ}_{SLOW}^{BF} {α}_{SMALL}^{BF} {m}^{BF}} {A}_{SMALL , i}^{BF} . \qquad ( 25 )$$

It is to be appreciated that the PwD categorizes and provides the size of meals as small, medium large meal amounts, as well as the meal speed. For instance, all small meals can be simply represented by an average value Ā_{SMALL}^{BF}. Thus, for example, all fast small meals may be represented by Equation (26) as:

λ_{FAST}^{BF}α_{SMALL}^{BF}m^{BF}Ā_{SMALL}^{BF} (26).

Collecting all the terms together, Equation (25) then can be rewritten as Equation (27) as:

$${\sum }_{i = 1}^{{α}_{SMALL}^{BF} {m}^{BF}} {K}^{BF} {A}_{SMALL}^{BF} = {α}_{SMALL}^{BF} {m}^{BF} ( {λ}_{FAST}^{BF} {K}_{FAST}^{BF} + {λ}_{REG}^{BF} {K}_{REG}^{BF} + {λ}_{SLOW}^{BF} {K}_{SLOW}^{BF} ) {\overline{A}}_{SMALL}^{BF} . \qquad ( 27 )$$

Now considering all the meal types we get the following relation shown by Equation (28) is follows:

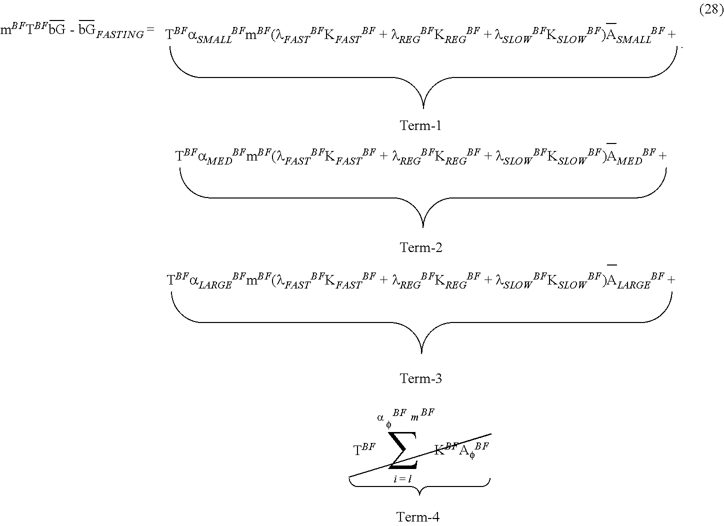

On further simplification, Equation (28) becomes: b̅G̅^{BF}− b̅G̅_{FASTING}=(λ_{FAST}^{BF}K_{FAST}^{BF}+λ_{REG}^{BF}K_{REG}^{BF}+λ_{SLOW}^{BF}K_{SLOW}^{BF})(α_{SMALL}^{BF}Ā_{SMALL}^{BF}+α_{MED}^{BF}Ā_{MED}^{BF}Ā_{MED}^{BF}+α_{LARGE}^{BF}Ā_{LARGE}^{BF})

The last group of terms on the right-hand side are the weighted amplitude terms which is the average amplitude. Thus, equation (28) can be further rewritten as: b̅G̅^{BF}− b̅G̅_{FASTING}=(λ_{FAST}^{BF}K_{FAST}^{BF}+λ_{REG}^{BF}K_{REG}^{BF}+λ_{SLOW}^{BF}K_{SLOW}^{BF}) Ā^{BF}. And (λ_{FAST}^{BF}K_{FAST}^{BF}+λ_{REG}^{BF}K_{REG}^{BF}+λ_{SLOW}^{BF}K_{SLOW}^{BF}) is a factor for given lifestyle characteristics. Similarly for other meals, relations can be derived, such as: b̅G̅^{LU}− b̅G̅_{FASTING}=(λ_{FAST}^{LU}K_{FAST}^{LU}+λ_{REG}^{LU}K_{REG}^{LU}+λ_{SLOW}^{LU}K_{SLOW}^{LU})Ā^{LU}, and b̅G̅^{SU}− b̅G̅_{FASTING}=(λ_{FAST}^{SU}K_{FAST}^{SU}+λ_{REG}^{SU}K_{SU}+λ_{SLOW}^{SU}K_{SLOW}^{SU})Ā^{SU}. So the final mean value of bG for a modal day can be defined according to Equation (29) as:

$$\overline{bG} = \frac{{T}^{FASTING}}{24} {\overline{bG}}_{FASTING} + \frac{{T}^{BF}}{24} {\overline{bG}}^{BF} + \frac{{T}^{LU}}{24} {\overline{bG}}^{LU} + \frac{{T}^{SU}}{24} {\overline{bG}}^{SU} . \qquad ( 29 )$$

The mean values b̅G̅^{BF}, b̅G̅^{LU} and b̅G̅^{SU} represent mean bG values for their corresponding meal sections. The above result Equation (29) shows that the specifics of the meal in the final meal equation collapse into a simple average relation in which the averages of an individual event is time weighted as is shown by Equation (29). The above conclusion according to the present disclosure was verified in simulation (FIG. 4). The relation provided by Equation (29) forms the basis to section the day as per lifestyle event and examine it from the perspective of replacing it by a meaningful average value. Alternatively, the mean value of bG for a modal day can be kept in its component parts such that the mean value for each component (e.g., b̅G̅_{Fasting}, b̅G̅^{BF}, b̅G̅^{LU} and b̅G̅^{SU}) can be utilized to determine the estimated HbAlC value for that particular component using the linear relationship between mean bG and estimated HbAlC as discussed herein. The estimated HbAlC values for each component may then be combined to determine the overall HbAlC for a given day according to Equation (30):

HbAlC_{Breakfast}+HbAlC_{Lunch}+HbAlC_{Supper}+HbAlC_{Fasting}=HbAlC (30).

While the above equation breaks down the estimated HbAlC values into time-based components (i.e., breakfast, lunch, supper and fasting) which combine into a complete day, the estimated HbAlC values may alternatively or additionally be broken into other variable-based components (e.g., event-based components or context-based components) as will become appreciated herein. For example, Equation (29) can be rewritten as Equations (29A) and (29B):

$$\begin{matrix}\overline{bG} = {\overline{bG}}_{FASTING} + \frac{{T}^{BF}}{24} ( {\overline{bG}}^{BF} - {\overline{bG}}_{FASTING} ) + \frac{{T}^{LU}}{24} ( {\overline{bG}}^{LU} - {\overline{bG}}_{FASTING} ) + \frac{{T}^{SU}}{24} ( {\overline{bG}}^{SU} - {\overline{bG}}_{FASTING} ) . \\ & ( 29 A ) \\ \overline{bG} = {\overline{bG}}_{FASTING} + \frac{{T}^{BF}}{24} Δ {\overline{bG}}^{BF} + \frac{{T}^{LU}}{24} Δ {\overline{bG}}^{LU} + \frac{{T}^{SU}}{24} Δ {\overline{bG}}^{SU} . & ( 29 B )\end{matrix}$$

Likewise, similar to Equations (29A) and (29B), Equation (30) can be rewritten as Equation (30A) in terms of HbAlC:

ΔHbAlC_{Breakfast}+ΔHbAlC_{Lunch}+ΔHbAlC_{Supper}+ΔHbAlC_{Fasting}=HbAlC (30A).

Another fundamental aspect to the algorithm is temporal weighting schema. The affect of past breakfast on current HbAlC is not equally weighted. Such weight schema is theoretically derived based on the assumption of lifespan of the erythrocytes. As discussed above, this approach can similarly extended to lipid profile, insulin profile, fructosamine and other metabolites

Temporal Weighting

Temporal weighting of bG values becomes relevant when the prediction model is derived between SMBG values and HbAlC. As mentioned previously above, each cohort has a finite life span of approximately 120 days. Thus, for this example, a lifespan of 120 days is considered. The aged cells are constantly being replaced by young erythrocytes. So at any given time each of the cohort's age will range from 0 to 119 days. Each cohort thus is exposed to a subset of corresponding bG data. Considering the glycated hemoglobin at current time and all the bG values over the last 120 days, then the bG value that is 120 days old influences only 1 out of 120 cohorts and none of the other cohorts with ages less than 120 days. On the other hand, the current bG value affects all ages of the surviving cohorts i.e. the last 120 cohorts. In context of constant bG for i^{th} day and considering the physiological aspect, this suggests that an appropriate weighted mean bG value can help improve HbAlC prediction.

In a simulation exercise, a lifespan L was set to 120 days and a number of cohorts N was made equal to 120 cohorts, where cohort # 120 is the oldest cohort, and cohort #1 is the newest. For a cohort aged L days (considering the oldest cohort), then the impact of bG, on HbAlC can be approximated according to Equation (31) as:

$$\frac{{\sum }_{i = 1}^{L} {bG}_{i} Δ T}{{\sum }_{i = 1}^{L} Δ T} = \frac{1}{L} {\sum }_{i = 1}^{L} {bG}_{i} , \qquad ( 31 )$$

where bGᵢ is glucose value on i^{th} day, where index i is 1, 2, 3, . . . L, from the latest glucose measurement to oldest glucose measurement. Similarly, for a cohort aged L-1 day, mean bG can be defined according to Equation (32) as:

$$\frac{{\sum }_{i = 1}^{L - 1} {bG}_{i} Δ T}{{\sum }_{i = 1}^{L - 1} Δ T} = \frac{1}{L - 1} {\sum }_{i = 1}^{L - 1} {bG}_{i} . \qquad ( 32 )$$

And so on. Collecting weights for same bG, the weights may be defined according to Equation (33) as:

$$\begin{matrix}[ ( \frac{1}{L} ) {bG}_{L} ( \frac{1}{L} + \frac{1}{L - 1} ) {bG}_{L - 1} … ( \frac{1}{L} + \frac{1}{L - 1} + … + \frac{1}{1} ) {bG}_{1} ] . & & \end{matrix} \qquad ( 33 )$$

It is to be appreciated that the above weighting scheme corresponds to a harmonic series. As such, the weights will be referred to herein as harmonic weighting.

FIG. 8 shows 4 weighting schemes that were considered in developing the prediction model for HbAlC. From using the harmonic weighting in the above Equation (33), it is clear that older bG values contribute progressively less and less to HbAlC value. If the area under the harmonic curve is considered, then the period covering 60 days represents 84.4% of the total, which is shown in Table 3.

TABLE 3
Area under the harmonic curve

| Visitation Period | Percentage Area |
|---|---|
| From = 1 To = 1 | 0 |
| From = 10 To = 1 | 28.1 |
| From = 20 To = 1 | 45.7 |
| From = 30 To = 1 | 59.0 |
| From = 40 To = 1 | 69.5 |
| From = 50 To = 1 | 77.8 |
| From = 60 To = 1 | 84.4 |
| From = 70 To = 1 | 89.6 |
| From = 80 To = 1 | 93.6 |
| From = 90 To = 1 | 96.5 |
| From = 100 To = 1 | 98.5 |
| From = 110 To = 1 | 99.6 |
| From = 120 To = 1 | 100 |

Additional results showed that harmonic temporal weighting is a relevant scheme in the determination of the HbAlC estimate based on SMBG measurements, and that the period over which SMBG data contributes significantly to estimating HbAlC is about 60 days (considering in this case life of erythrocytes as 120 days. Similar reasoning can be used when considering erythrocytes for other ages). Analysis results also supports that collecting bG values collected over a visitation period of about approximately 60 days provides the best estimate on HbAlC. In one embodiment, the collecting of both bG measurements and associated context of the bG measurement at daily times specified by the structured sampling schema is over a period of about 2 to about 4 months. In another embodiment, a small time window such as ranging from 1 week to 4 weeks can be used as a representative of glucose behavior covering a 3 to 4-month period. This allows the HCP and patient to revise the current therapy or behavior to try achieving prescribed targeted goals. The resulting predicted HbAlC then represents a future HbAlC which provides the patient and/or HCP the future glycemic level is assuming the current glucose behavior is maintained. The principles behind the process used to derive a correlation coefficient for meal sections according to the present disclosure is now discussed hereafter.

Correlation Coefficient for Meal Sections

Glucose data collected during two independent clinical studies in 2003 and 2006 were used to determine a correlation coefficient for meal sections from which to devise a sampling schema for use with the HbAlC prediction model. The clinical trials studied the post prandial glucose control for meals with different meal composition. The key aspects of each of the two studies are summarized below.

Meal Study 2003
1. Study was conducted during 2003-2004. The study was designed to examine the meal response of fixed insulin bolus to meals with varying glucose absorption characteristics.
2. Demographics of the subjects participating in the study are:
   a. Number of Subjects=23
   b. Number of study blocks=4
   c. Number of males=12, Number of females=11
   d. Age (40±9) years
   e. Weight (75±15) kg
   f. BMI (24.6±2.5) kg/m²
   g. HbAlC (7.0±1.0) %
3. Each visit is 4 days long:
   a. Day 1:
      i. Subject arrives in the evening to be instrumented.
      ii. Has an evening supper
      iii. Spot monitoring
   b. Day 2:
      i. 9 am Test meal (A, B, C, D, E and F)
      ii. 3 pm late lunch meal
      iii. 7 pm supper
   c. Day 3:
      i. 9 am Test meal (A, B, C, D, E and F)
      ii. 3 pm late lunch meal
      iii. 7 pm supper
   d. Day 4:
      i. Subject leaves around breakfast time
4. Number of study blocks is 4. A study block is the re-visitation of the subject for performing the meal study with a different test meal and/or insulin therapy algorithm.
5. Meal sections were extracted from Meal Study 2003. The sections were of duration:
   a. 6 hr, all test meals (@ 9:00 am)
   b. 4 hr, all late lunch (@ 3:30 pm)
   c. 8 hr, all supper (@ 7:00 pm)

### Meal Study 2006

1. Study was conducted during the year 2006-2007

2. Demographics of the subjects:
a. Number of Subjects=12
b. Number of study blocks=4
c. Number of males=7, Number of females=5
d. Age (45±9) years
e. Weight (75±14) kg
f. BMI (24.7±3.0) kg/m²
g. HbAlC (6.9±0.8) %

3. Each visit (block) is 4 days long:
a. Day 1:
   i. Subject arrives in the evening to be instrumented.
   ii. Has a evening supper
   iii. Spot monitoring
b. Day 2:
   i. 9 am Test meal (A, B, E and F)
   ii. 3 pm late lunch meal
   iii. 7 pm supper
c. Day 3:
   i. 9 am Test meal (A, B, E and F)
   ii. 3 pm late lunch meal
   iii. 7 pm supper
d. Day 4:
   i. Subject leaves around breakfast time

4. Number of study blocks is 4. A study block is the re-visitation of the subject for performing the meal study with a different test meal and/or insulin therapy algorithm.

5. Meal sections were extracted from Meal Study 2006. The sections were of duration:
a. 6 hr, all test meals (@ 9:00 am)
b. 4 hr, all late lunch (@ 3:30 pm)
c. 8 hr, all supper (@ 7:00 pm)

The above test meal labels A-F describe the meal speed. Meals labeled A and B are fast meals, meals labeled C and D are regular, and meals labeled E and F are slowly absorbing meals. The meals were classified by a professional dietician. The meal study data set provided discrete frequently measured bG data, where the sampling rates for the time window covering the test meals were 10 minutes. Sampling rates at other times range from 1 minute to as rarely as hourly measurement, such as overnight. Also available in the bG data is specific insulin, ingested mixed meal information and interventions. It is to be appreciated that the clinical bG data set did not include HbAlC values. HbAlC values were then generated artificially by using the Mortensen model (Equations (1)-(3)) with the bG data.

It is clear from earlier analysis that there exists a linear relationship between true mean bG and HbAlC. It is then clear that one could simply focus on the question of determining either true mean bG or HbAlC. Given the continuous and/or frequent bG measurements in the bG data, the bG curves were then sectioned into relevant groups and correlation between various parameters such as minimum, maximum, glucose value at specified time and so forth were correlated to true mean bG as well as HbAlC.

In regards to HbAlC, this value was determined by inputting the glucose curve to the Mortensen model Equations (1)-(3). In this regard then, the meal data was first divided into meal sections. Each of the meal sections was curve fitted and then the resulting signal was repeated to create as input a bG input signal of duration 150 days. The resulting profile was then passed through the Mortensen model. (Equations (1)-(3)) to generate the HbAlC values. In this way, HbAlC for each of the meal sections was generated.

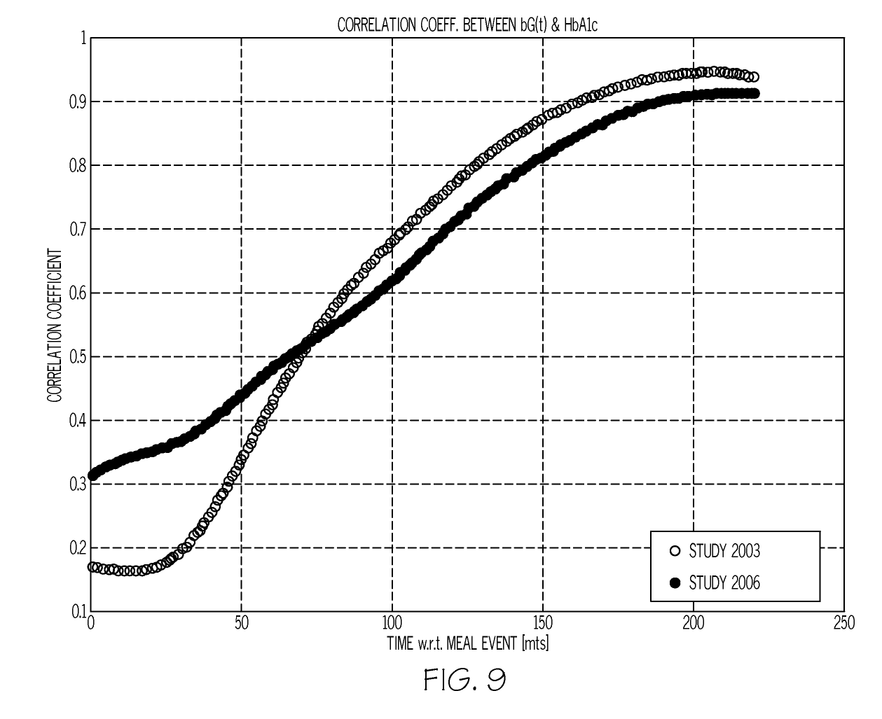

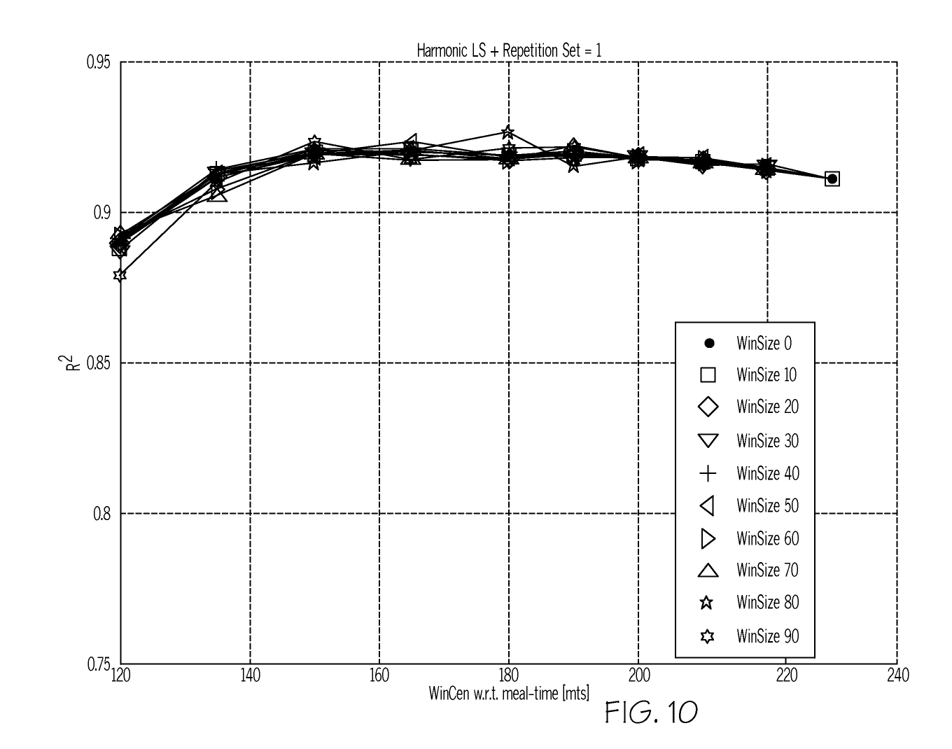

Next, several predictors were examined to correlate with HbAlC. The most meaningful single-point predictor discovered by the inventors was a bG measurement taken at a particular post-prandial time point. For this predictor, a Pearson correlation coefficient was used as a function of bG(t) which is shown plotted in FIG. 9. The correlation coefficient for meal sections extracted from the clinical meal studies of 2003 and 2006 shows that there is a strong linear relationship between HbAlC and post prandial bG measurement when t>150 minutes.

Although the correlation coefficients may differ for different clinical studies, in general the trends are expected to be similar. As shown by FIG. 9, a low correlation is seen in the 1^{st} hour; the correlation then starts increasing and reaches values greater than 0.8 for 2.5 hrs postprandial. Such variation could be explained by meal type, meal amount and associated insulin therapy. Low correlation in early hours of postprandial is due to transients caused by variations due to meal glucose absorption and due to insulin absorption characteristics. As the transients die out the correlation increases. The increased correlation for the clinical studies is also due to following reasons: the subjects are well motivated, so in general, their glycemic excursion should recover quite consistently for subjects during postprandial period.

The variations in meal behavior are due to main factors such as physiology, meal content variation, inaccuracies in physiological parameter estimates, basal setting. The correlation coefficients indicate that meals correlate to HbAlC very strongly when bG measurements are conducted postprandially in time range around 3 hours. It is also clear from simulation that the transient bG has comparatively less impact than the steady state behavior of the meal that is the relatively slow and steady push. The variability in the early transients is clearly indicative of lack of specific knowledge of day to day physiological variability and imprecise knowledge of meal but the general control strategy on the latter post prandial state is important in achieving low HbAlC. While this method was described with respect to HbAlC, the general method also provides in detail how one can extend the approach to cover other biological values (e.g., metabolites and biomarkers) such as fructosamine (a biomarker for glycemia over past 3˜4 weeks, where fructosamine is glycated albumin). Furthermore, solutions for problems under different assumptions can be redone to derive estimation relations and/or the parameters.

The following section hereafter focuses on deriving an optimal sampling schema for determination of true mean bG and HbAlC. Sampling schema is determined by using the equations developed in earlier sections, such as lifestyle related time weighting addressing a modal day, and glucose weighting addressing the data covering a visitation period (i.e., period between visitations).

Structured Sampling Schema

Using clinical data from Meal study 2003, bG profiles are generated by combining various meal sections by randomly selecting bG profile sections from different meal bins and concatenating the sections. The various meal bins are listed in Table 4.

TABLE 4
Meal Bins

| | Breakfast | Lunch | Supper + Overnight |
|---|---|---|---|
| Low - HbA1C | First ⅓^{rd} of ranked | First ⅓^{rd} of ranked | First ⅓^{rd} of ranked |
| | Breakfast meals | lunch meals | supper meals |
| Medium - HbA1C | Second ⅓^{rd} of | Second ⅓^{rd} of ranked | Second ⅓^{rd} of ranked |
| | ranked Breakfast | lunch meals | supper meals |
| | meals | | |
| High - HbA1C | Third ⅓^{rd} of | Third ⅓^{rd} of ranked | Third ⅓^{rd} of ranked |
| | ranked Breakfast | lunch meals | supper meals |
| | meals | | |

The bins in Table 4 represent meal sections and are first of all grouped by collecting the sections obtained from breakfast, lunch and supper and overnight time periods. The meal sections were further ranked and sorted in ascending order in terms of corresponding HbAlC values from simulation. The breakfast meal pool was then divided into 3 equal groups by selecting the first one-third breakfast meals and labeled as low—HbAlC, then the second one third of breakfast meals labeled as Medium—HbAlC and the remaining breakfast meals as High—HbAlC. In a similar fashion, lunch and supper are also binned. In all, 9 meal bins were created. To create lifestyle based bG sequence, lifestyle is described as the modal day consisting of breakfast starting at 8 am with one of the HbAlC group (Low, Medium or High); lunch at noon with one of the HbAlC group (Low, Medium or High) and supper at 6 pm with one of the HbAlC group (Low, Medium or High). In this manner, 174 bG sequences were generated covering various combinations.

As mentioned in the previous section, if bG measurements are conducted postprandially around the time interval when the correlation coefficient is high (e.g., t>150 minutes, FIG. 9) a good estimate of HbAlC can be anticipated. Therefore, the key factors relating to a useable prediction of HbAlC from a series of bG measurements are the following: (a) timing of bG measurement, (b) number of bG measurements (in range of 2-6 measurements per day), (c) accuracy of predicted AlC, and (d) bias of the predicted AlC.

As per lifestyle (primarily done for meal in the illustrated embodiment) the sampling schema was setup according to Table 5 as follows:

TABLE 5
Sampling Schema setup

| | |
|---|---|
| Center of the Sampling | Determine the optimal expected time at |
| window (WinCen) | which one should sample for SMBG. |
| Size of the sampling window | The allowance/tolerance to measurement |
| (WinSize) | time window around WinCen. |
| Number of Days (nDays) | SMBG data is collected over period of last |
| | nDays days. |
| Number of samples (nSamples) | The bG sampling is event driven. With |
| | respect to each meal event, the number of |
| | samples collected during the specified |
| | number of days, nDays. As an example |
| | nSamples = 50 means that as described by |
| | Lifestyle (FIG. 5) for breakfast there are 50 |
| | bG measurements spanning over nDays, |
| | then 50 measurements for lunch spanning |
| | the nDays and then for supper 50 |
| | measurements spanning the nDays. |
| $Sampling Ratio , \frac{nSamples}{nDays}$ | It is the ratio of number of glucose measurements to the number of days (nDays) over which the samples are collected, for each event type. For example, nSamples = 50 and nDays = 70 then Sampling ratio = 50/70. |

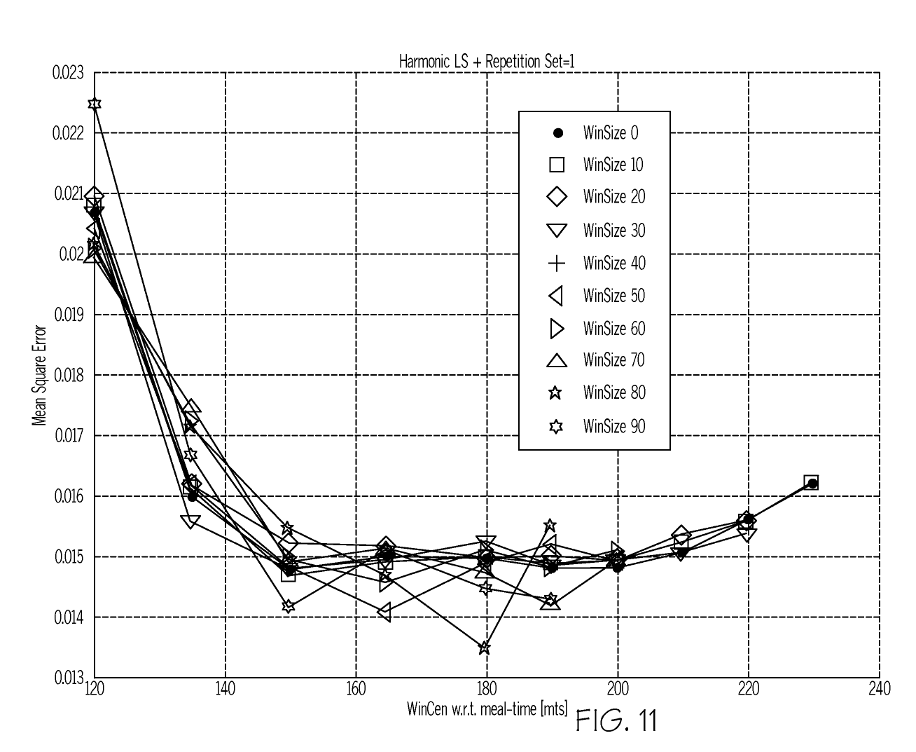

Linear regression was then carried out to predict HbAlC from SMBG measurements, whereby SMBG values were processed by various lifestyle weighting and averaging strategies. FIG. 10 shows the R² (R-squared value) from the linear regression against HbAlC for various values of WinCen and WinSize for the lifestyle. For the illustrated plot of FIG. 10, the visitation period (nDays) equals 60 days, and the number of samples (nSamples) also equals 60. As shown, the best R² is centered on post-prandial time of 190 minutes. Similarly plotting the results for mean squared error (FIG. 11) it is observed that a postprandial measurement around 180 minutes provides minimum error in HbAlC estimate.

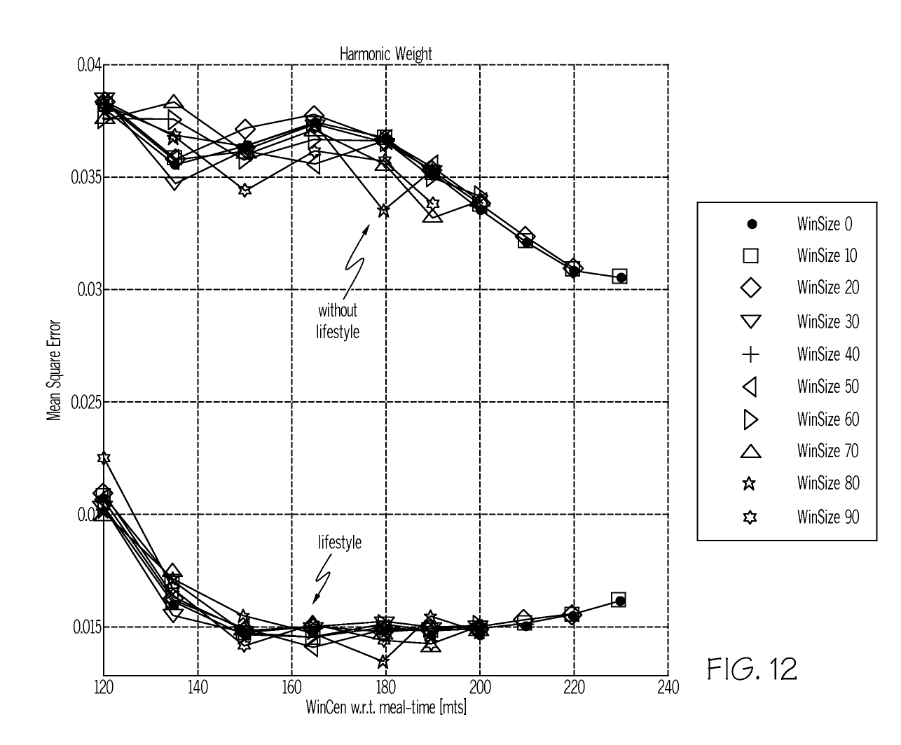

In FIG. 12, a comparison between the daily lifestyle weighting and no daily lifestyle weighting shows that daily life style weighting produces lower MSE (mean squared error).

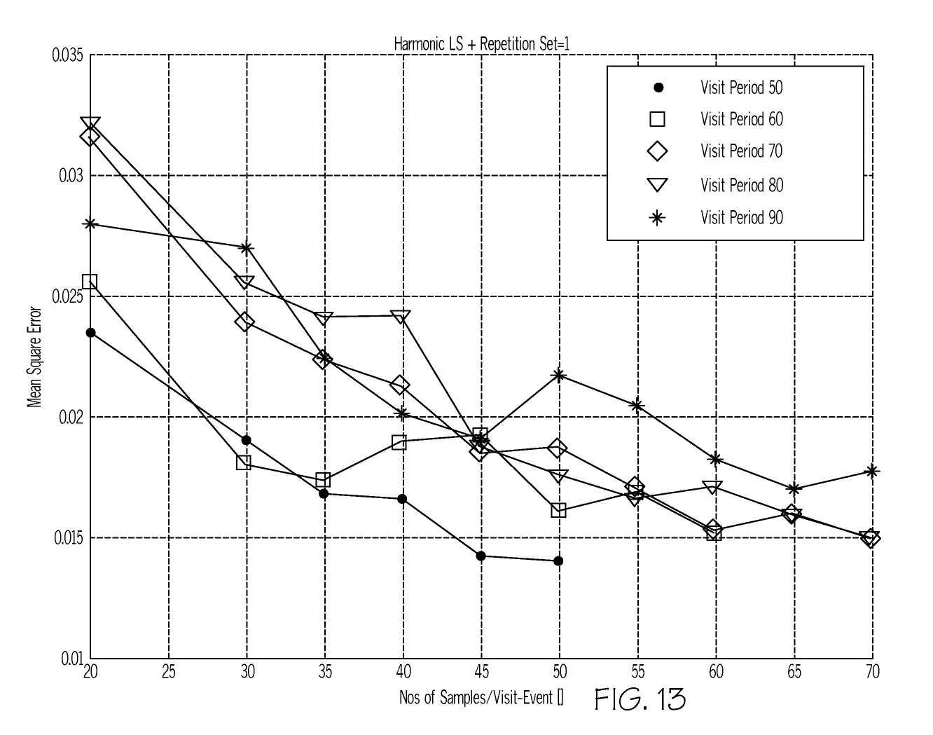

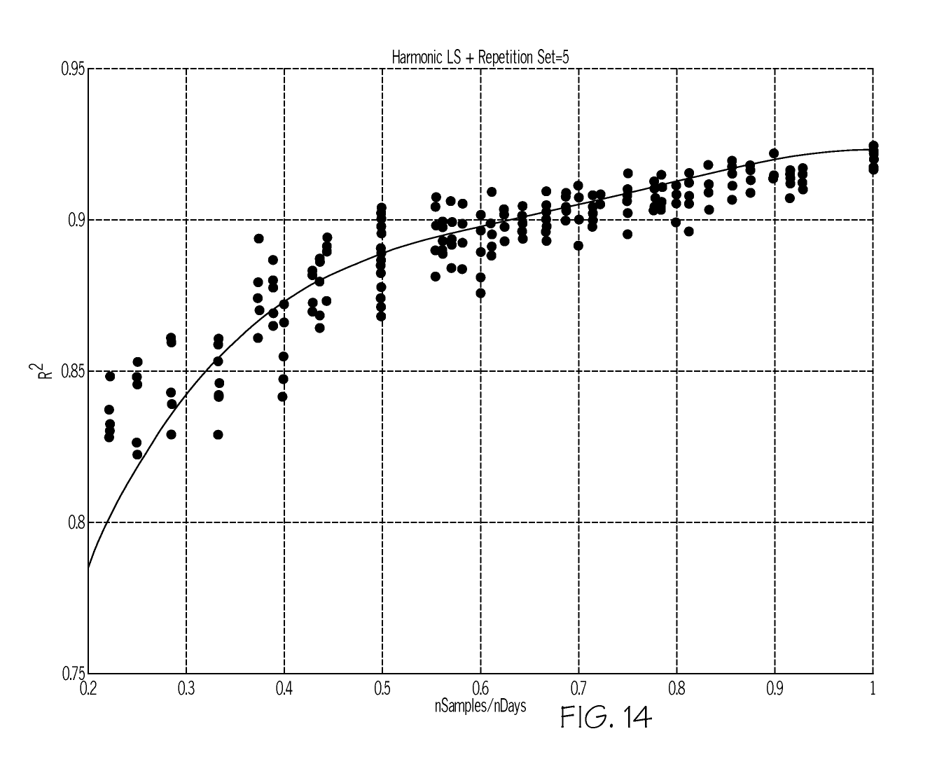

FIG. 13 shows the impact of nDays (visitation period) and nSamples for WinCen of 190 minutes and WinSize of 50 minutes. It shows that MSE reduces as the number of SMBG measurements is increased. In particular, there is an impact of nDays. The number of days shows that there are an optimal number of days beyond which the R² does not improve. To determine the best nSamples and nDays one actually needed to look at the sampling ratio which is the number of samples per event (nSamples/nDays). The requirement was to have the ratio as small as possible with some acceptable R². What was observed was that below 0.5 both R² deteriorate at a rapid pace and also the spread in their values became greater. For a sampling ratio greater than 0.5 and above, the R² value was >0.85. For R²>0.9, a sampling ratio

$$\frac{nSamples}{nDays}$$

of 0.55 was obtained as shown by FIG. 14.

Regression Model for Estimating HbAlC

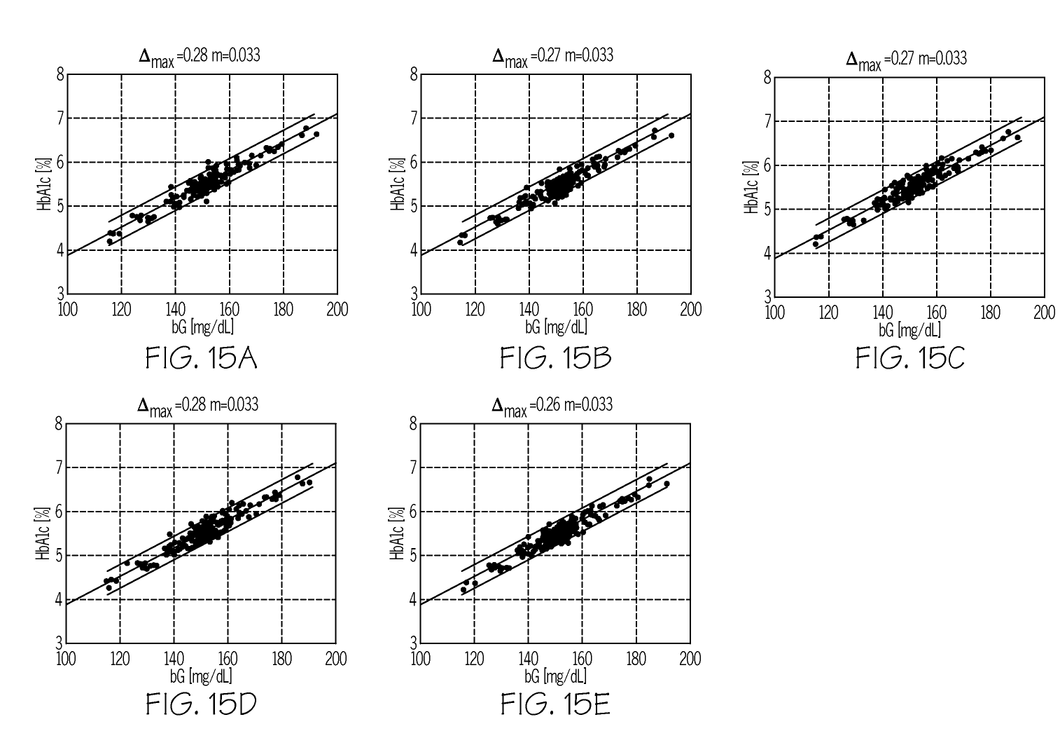

As per the sampling schema, the sampled bG data were then regressed and plotted, which are shown by FIGS. 15A-E. Tabulated results of the regression are provided in Table 6, which shows that the parameters for each linear regression are in close proximity to each other. In FIGS. 15A-E, the prediction line (centerline) and a 95% confidence interval (CI) boundaries (above and below curves) are shown in the subplots. The 95% CI covers a range which deviates approximately 0.26% HbAlC from the nominal value.

TABLE 6
Linear regression parameters

| FIG. | Delta | Slope | Intercept |
|---|---|---|---|
| 15A | 0.28 | 0.033 | 0.587 |
| 15B | 0.27 | 0.033 | 0.581 |
| 15C | 0.27 | 0.033 | 0.588 |
| 15D | 0.28 | 0.033 | 0.547 |
| 15E | 0.26 | 0.033 | 0.548 |

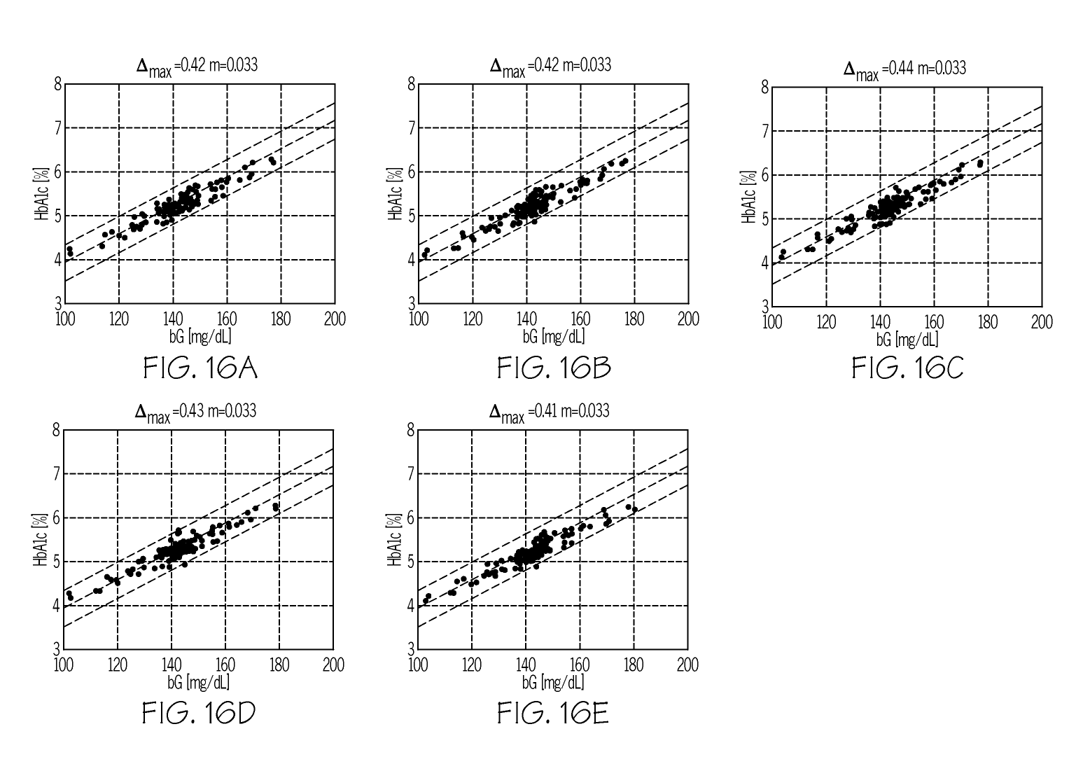

FIGS. 16A-E show that CI boundaries contain almost all of the HbAlC observations. A slope of 0.033 or 1/30 is obtained from the linear regression. In summary, the optimal SMBG sampling parameters along with regression parameters for determining an estimated true mean bG value and estimated HbAlC value is listed in Table 7.

TABLE 7
Structured Sampling Schema

| Parameter | Optimal value |
|---|---|
| WinCen | 190 min |
| WinSize | 50 min |
| nSamples | 45 samples per event |
| nDays | 80 days |
| Weighting function | Harmonic |
| Lifestyle weighting | Yes |
| Estimated HbA1C | 0.033 b̅G̅ + 0.5702 |

The Estimated HbAlc in Table 7 comprises virtual HbAlc determined based of the Mortenson model in which patient specific relationships and estimates can be addressed by calibration as discussed later herein. Alternatively, the weighted component based bG estimates presented herein can be used in HbAlc estimate relations such as the Nathan's relation (Nathan, D. M.; Schoenfeld, D.; Kuenen, J.; Heine, R., J.; Borg, R.; Zheng, H.; “Translating the AlC Assay into estimated average glucose values,” Diabetes Care, Vol 31, Nos 8, August 2008, pp. 1-6.), which estimate patient HbAlc, or Abensour's relation (U.S. Patent Publication No. US 2007/0010950 A1).

Validation

To validate the results obtained above in Table 7, which were derived using the meal sections extracted from the Meal Study 2003, the Meal Study 2006 was then used. Similar to Meal Study 2003, all the meal sections from Meal Study 2006 were extracted. Overall, 286 meal sections were obtained from the 2006 study. All the meal sections were then fitted by a polynomial curve, and ordered in an ascending order by their individual HbAlC values (obtained by using the Mortensen Model). The meal sections were then binned into groupings done in the manner explained for Meal Study 2003. Using the meal sections, a bG sequence covering a duration of 300 days was generated as per the previous lifestyle used in the 2003 meal study. The simulation duration was also set to 300 days. The bG profiles and HbAlC were then stored for sampling and HbAlC prediction. In all 108 simulations were generated.

The bG values were then sampled as per the Sampling Schema listed in Table 7. Using the sampled bG values for each of the 108 simulation cases, mean bG was determined. The relation between the mean bG and HbAlC, as determined by simulation, is plotted in FIGS. 16A-E. Each of the FIGS. 16A-E is a subplot which simply repeats a random sampling as per the schema explained earlier. Each subplot shows the estimated mean bG with the true HbAlC. The upper and lower lines in each subplot indicated 3 standard deviations (SD line) from the predicted HbACl algorithm, 0.033 b̅G̅+0.5702, (e.g., center line in each subplot) as determined previously above. It is to be appreciated that the distance between the SD line and the mean behavior on an average is 0.44% HbAlC. Therefore, as expected there was a degradation (spread) in the estimated HbAlC value, however the resulting precision was still within 3% CV.

From the above results, if a slightly lower R² of 0.85 is used, then the number of measurements/event can be reduced to 45 over 80 days. With 3 meal events per day plus a nighttime measurement, then the number of measurements equal 180 measurements. This implies approximately 2.25 measurements/day are needed as per sampling schema described above to achieve an estimated HbAlC value that has a precision within 3% CV.

It should be appreciated that the stated derived results are one of many ways of using the approach. The estimated value could alternatively or additionally be plugged into pre-defined models, for instance Nathan's relation as described in Nathan, D. M.; Schoenfeld, D.; Kuenen, J.; Heine, R., J.; Borg, R.; Zheng, H.; “Translating the AlC Assay into estimated average glucose values,” Diabetes Care, Vol 31, Nos 8, August 2008, pp. 1-6., where the mean bG value based of the analysis presented herein is used in the other model. Nathan's relation, for example, can provide an estimate through a relationship that that is accepted in the medical community while using a better estimate of the mean bG (which should ideally improve the estimate as well as provide better acceptance of the result by the medical community).

Implementation Examples

The above described sampling schema and prediction algorithm for providing both an estimated true mean blood glucose value and an estimated glycated hemoglobin (HbAlC) value from structured spot measurements of blood glucose may be implemented using hardware, software or a combination thereof. For example, the above described sampling schema and prediction algorithm may be implemented in one or more microprocessor based systems, such as a portable computer or other processing systems, such as personal digital assistants (PDAs), or directly in self-monitoring glucose devices or meters (bG meters) equipped with adequate memory and processing capabilities to process a chronological sequence of measurements of a time dependent parameter measured in or on the human body, namely of the glucose level (e.g. the glucose (bG) level). In some embodiments, remote servers may process the measurements to determine the estimated and/or predicted values and provide these determined values to a personal glucose meter, PDA or the like. In these embodiments, the personal glucose meter, PDA or the like may thereby be operable with a relatively smaller processor that could not as quickly determine the values compared to an application running on the remote server.

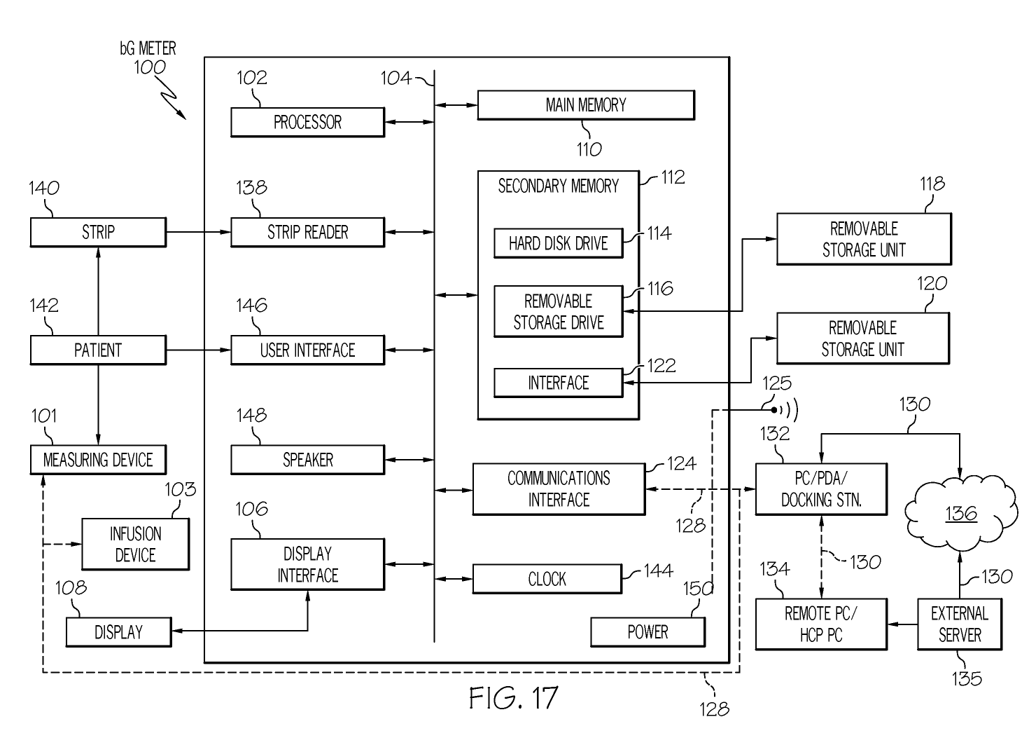

In an example embodiment, the sampling schema and prediction algorithm are implemented in software running on a self-monitoring blood glucose (bG) meter 100 as illustrated in FIG. 17. The bG meter 100 is common in the industry and includes essentially any device that can function as a glucose acquisition mechanism. The bG meter 100 or acquisition mechanism, device, tool, or system includes various conventional methods directed toward drawing a sample (e.g. by finger prick) for each test, and making a spot determination of the glucose level using an instrument that reads glucose concentrations by optical, electrochemical, electromechanical or calorimetric detection/measurement methods. In addition, the bG meter 100 may include and/or communicate with measuring devices 101 capable of measuring one or more biological measurement (e.g., glucose, lipids and/or triglycerides. For example, measuring devices the bG meter 100 can include and/or communicate with devices with indwelling catheters and subcutaneous tissue fluid sampling devices (e.g., a continuous glucose monitor (CGM) device) and/or a drug pump/infusion device 103.

In the illustrated embodiment, the bG meter 100 includes one or more microprocessors, such as processor 102, which is connected to a communication bus 104, which may include data, memory, and/or address buses. The bG meter 100 may include a display interface 106 providing graphics, text, and other data from the bus 104 (or from a frame buffer not shown) for display on a display 108. The display interface 106 may be a display driver of an integrated graphics solution that utilizes a portion of main memory 110 of the bG meter 100, such as random access memory (RAM) and processing from the processor 102 or may be a dedicated graphics card. In another embodiment, the display interface 106 and display 108 additionally provide a touch screen interface for providing data to the bG meter 100 in a well-known manner.

Main memory 110 in one embodiment is random access memory (RAM), and in other embodiments may include other memory such as a ROM, PROM, EPROM or EEPROM, and combinations thereof. In one embodiment, the bG meter 100 includes secondary memory 112 which may include, for example, a hard disk drive 114 and/or a removable storage drive 116, representing a floppy disk drive, a magnetic tape drive, an optical disk drive, a flash memory, etc. The removable storage drive 116 reads from and/or writes to a removable storage unit 118 in a well-known manner. Removable storage unit 118, represents a floppy disk, magnetic tape, optical disk, flash drive, etc. which is read by and written to by the removable storage drive 116. As will be appreciated, the removable storage unit 118 includes a computer usable storage medium having stored therein computer software and/or data.

In alternative embodiments, secondary memory 112 may include other means for allowing computer programs or other instructions to be loaded into the bG meter 100. Such means may include, for example, a removable storage unit 120 and an interface 122. Examples of such removable storage units/interfaces include a program cartridge and cartridge interface, a removable memory chip (such as a ROM, PROM, EPROM or EEPROM) and associated socket, and other removable storage units 120 and interfaces 122 which allow software and data to be transferred from the removable storage unit 120 to the bG meter 100.

The bG meter 100 in one embodiment includes a communications interface 124. The communications interface 124 allows software and data to be transferred between the bG meter 100 and an external device(s) 132. Examples of communications interface 124 may include one or more of a modem, a network interface (such as an Ethernet card), a communications port (e.g., USB, firewire, serial or parallel, etc.), a PCMCIA slot and card, a wireless transceiver, and combinations thereof. In one embodiment, the external device 132 is a personal computer (PC), and in another embodiment is a personal digital assistance (PDA). In still another embodiment, the external device 132 is a docking station wherein the communication interface 124 is a docket station interface. In such an embodiment, the docking station may be provided and/or connect to one or more of a modem, a network interface (such as an Ethernet card), a communications port (e.g., USB, firewire, serial or parallel, etc.), a PCMCIA slot and card, a wireless transceiver, and combinations thereof. Software and data transferred via communications interface 124 are in the form of wired or wireless signals 128 which may be electronic, electromagnetic, optical, or other signals capable of being sent and received by communications interface 124. For example, as is known, signals 128 may be sent between communication interface 124 and the external device(s) 132 using wire or cable, fiber optics, a phone line, a cellular phone link, an RF link, an infrared link, other communications channels, and combinations thereof. In some embodiments, the bG meter 100 comprises remote server connection 125 to send data to an external server such that the external server processes the requested information and sends the results back to the bG meter 100 as discussed herein.

In one embodiment, the external device 132 is used for establishing a communication link 130 between the bG meter 100 and still further electronic devices such as a remote Personal Computer (PC) of the patient, and/or a health care provider (HCP) computer 134, or an external server 135 directly or indirectly, such as through a communication network 136, such as the Internet and/or other communication networks. The communication interface 124 and/or external device(s) 132 may also be used to communicate with further data gathering and/or storage devices such as insulin delivering devices, cellular phones, personal digital assistants (PDA), etc. Specific techniques for connecting electronic devices through wired and/or wireless connections (e.g. USB and Bluetooth, respectively) are well known in the art.

In the illustrative embodiment, the bG meter 100 provides a strip reader 138 for receiving a glucose test strip 140. The test strip 140 is for receiving a sample from a patient 142, which is read by the strip reader 138. Data, representing the information provided by the test strip, is provided by the strip reader 138 to the processor 102 which executes a computer program, e.g., provided in main memory 110, to perform various calculations as discussed in great detail below on the data. The results of the processor 102 from using the data is displayed on the display 108 and/or recorded in secondary memory 112 by the processor 102, which is herein referred to as self-monitored glucose (bG) data. The bG data may include, but not limited thereto, the glucose values of the patient 142, the insulin dose values, the insulin types, and the parameter values used by processor 102 to calculate future glucose values, supplemental insulin doses, and carbohydrate supplements. Each glucose value and insulin dose value is stored in memory 112 by the processor 102 with a corresponding date and time. An included clock 144 of the bG meter 100 supplies the current date and time to processor 102. The bG meter 100 further provides a user input device(s) 146 such as keys, touchpad, touch screen, etc. for data entry, program control, information requests, and the likes. A speaker 148 is also connected to processor 102, and operates under the control of processor 102 to emit audible and/or visual alerts/reminders to the patient of daily times for bG measurements and events, such as for example, to take a meal, of possible future hypoglycemia, and the likes. A suitable power supply 150 is also provided to power the bG meter 100 as is well known to make the meter portable.

The terms “computer program medium” and “computer usable medium” are used to generally refer to media such as removable storage drive 116, a hard disk installed in hard disk drive 114, signals 128, etc. These computer program products are means for providing software to bG meter 100. Embodiments of this disclosure include such computer program products.

Computer programs (also called computer control logic) are stored in main memory 110 and/or secondary memory 112. Computer programs may also be received via the communications interface 124. Such computer programs, when executed, enable the bG meter 100 to perform the features of the present disclosure as discussed herein. In particular, the computer programs, when executed, enable processor 102 to perform the functions of the present disclosure. Accordingly, such computer programs represent controllers of bG meter 100. Alternatively or additionally, the computer programs may be stored and/or run on remote servers with input and output data communicated via wired or wireless communication networks.

In an embodiment where the disclosure is implemented using software, the software may be stored in a computer program product and loaded into bG meter 100 using removable storage drive 116, removable storage unit 120, hard disk drive 114, or communications interface 124. The control logic (software), when executed by the processor 102, causes the processor 102 to perform the functions of the disclosure as described herein.

In another embodiment, the disclosure is implemented primarily in hardware using, for example, hardware components such as application specific integrated circuits (ASICs). Implementation of the hardware state machine to perform the functions described herein will be apparent to persons skilled in the relevant art(s).

In yet another embodiment, the disclosure is implemented using a combination of both hardware and software.

In an example software embodiment of the disclosure, the methods described hereafter are implemented in the C++ programming language, but could be implemented in other programs such as, but not limited to, Visual Basic, C, C#, Java or other programs available to those skilled in the art (or alternatively using script language or other proprietary interpretable language used in conjunction with an interpreter).

As mentioned above, the bG meter 100 is used by the patient 142 for recording, inter alia, insulin dosage readings and spot measured glucose levels. Such bG data obtained by the bG meter 100 in one embodiment is transferable via the communication interface 124 to another electronic device, such the external device 132 (PC, PDA, or cellular telephone), or via the network 136 to the remote PC and/or HCP computer 134. Examples of such bG meters include but are not limited to, the Accu-Chek Active meter and the Accu-Chek Aviva system both by Roche Diagnostics, Inc., which are compatible with the Accu-Chek 360° Diabetes management software to download test results to a personal computer or the Accu-Chek Pocket Compass Software for downloading and communication with a PDA. The program may run on a remote server and generate result. The result is available by one or more communication mode(s) stated earlier. The program device is also functional with 3^{rd} party devices which communicate with 132, 134. Examples of communicating and exchanging information between various devices is further provided in more detail in commonly owned U.S. application Ser. No. 12/119,143, which is herein incorporated by reference.

Accordingly, it is to be appreciated that the bG meter 100 includes the software and hardware necessary to process, analyze and interpret the self-recorded diabetes patient (i.e., bG) data in accordance with predefined flow sequences (as described below in detail) and generate an appropriate data interpretation output. In one embodiment, the results of the data analysis and interpretation performed upon the stored patient data by the bG meter 100 are displayed in the form of a report, trend-monitoring graphs, and charts to help patients manage their physiological condition and support patient-doctor communications. In other embodiments, the bG data from the bG meter 100 may be used to generated reports (hardcopy or electronic) via the external device 132 and/or personal computer (PC) and/or HCP computer 134.

The bG meter 100 further provides the user and/or his or her HCP with the possibilities of a) editing data descriptions, e.g., the title and description of a record; b) saving records at a specified location, in particular in user-definable directories as described above; c) recalling records for display; d) searching records according to different criteria (date, time, title, description, context information, data indexing, time range, etc.); e) sorting records according to different criteria (values of the bG level, date, time, duration, title, description etc.); f) deleting records; g) exporting records; and/or h) performing data comparisons, modifying records, excluding records as is well known. Alternatively or additionally, these functions may be performed on an external device 132, a HCP computer 134 or an external server 135.

As used herein, lifestyle is described in general as a pattern in an individual's habits such as meals, exercise, and work schedule. The individual additionally may be on medications such as insulin therapy or orals that they are required to take in a periodic fashion. Influence of such action on glucose is implicitly considered by the present disclosure.

Estimating True Mean bG and HbAlC

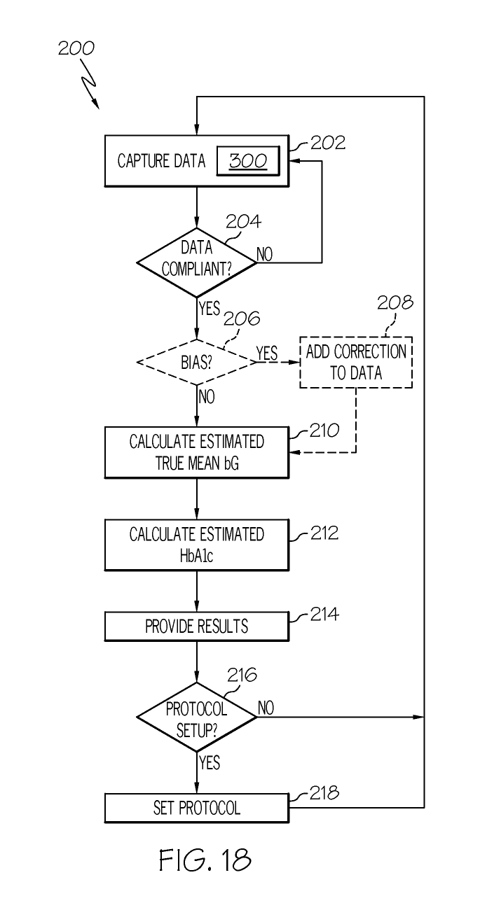

With reference made also to FIG. 18, a method 200 according to one embodiment of the present disclosure is described. In step 202, bG (i.e., spot) measurements of the patient 142 is captured. In one embodiment, each of the bG spot measurements is captured via strip 140 provided with a sample of the patient's which is then in turn read by a strip reader and analyzed by processor 102 to give the bG measurement of the patient 142. In other embodiments, the bG measurements may be captured at times dictated by the continuous glucose monitor 101 or other bG measuring devices and/or as commanded by the patient. As is well know the result of a newly taken bG measurement is displayed to the patient on display 108 as well as stored such as, for example, in memory 112 together with a time (e.g., GMT) and date of the measurement, via processor 102 reading clock 144 in step 202.

In one embodiment and generally, as mentioned above the bG meter 100 stores the results of the glucose (bG) measurements in its memory 112 together with a date-time stamp and associated event information (i.e., information regarding the context in which the measurement was obtained) to create a chronological sequence or set G of bG spot measurements, such as measurements bG₁^{k}, bG₂^{k}, bG₃^{k}, bG₄^{k}, and bG₅^{k}, where k is the day. The measurement set G is sorted by increasing time and may span several days. In one embodiment, the date stored in memory with the measurement consists of some representation of day/month/year, and the time consists of some representation of the time of day (e.g. hh:mm:ss). In other embodiments, other date and time recording methods may be used, such as for example, using a Julian calendar and an alternative count interval for time.

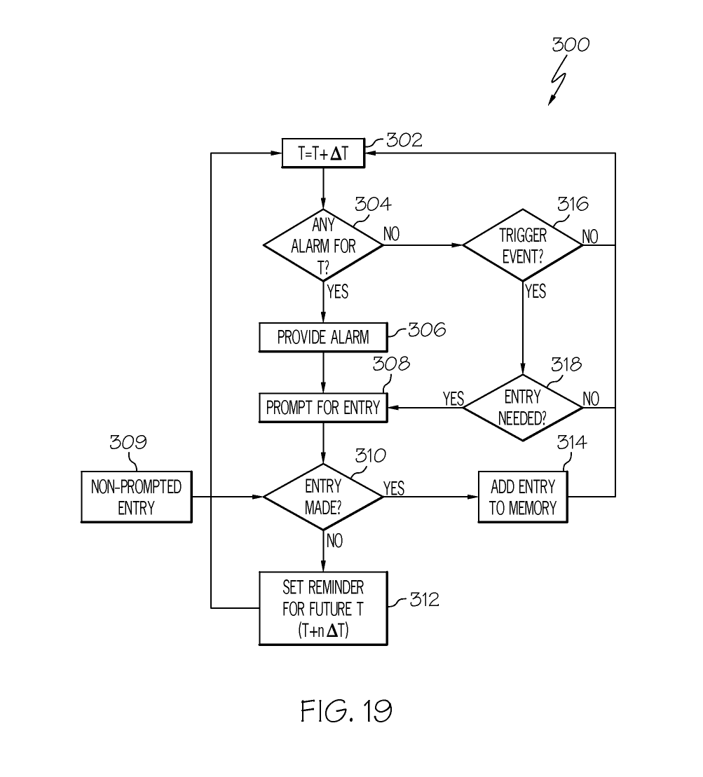

Along with each bG measurement, the patient is requested to input event information concerning the patient's lifestyle. In one embodiment, the meter 100 has enough memory to maintain bG data for at least 40-80 days with the associated event information concerning the patient's lifestyle. In another embodiment, the meter 100 has enough memory to maintain bG data for at least weeks with the associated event information concerning the patient's lifestyle. In one embodiment, lifestyle is classified by information concerning the following events: breakfast, lunch, supper, snack, exercise, physical activity, stress, alternate state, medication and optionally any other relevant event that is custom set into the meter. As with the bG measurements, such events are time stamped and associated with a description of event such as, for example, magnitude, intensity, duration, etc. Other such event characterizations are described more fully in commonly owned U.S. application Ser. Nos. 11/297,733 and 12/119,201, which are herein incorporated by reference. Manual input of the event description by the patient in one embodiment is driven by a questionnaire presented to the patient on the meter 100. In one embodiment, the questionnaire is provided by an HCP or designed to be set up by the patient according to provided instructions contained on the meter 100. In another embodiment, the meter 100 is provided with scheduled reminders (e.g., alarms for taking medication) which are provided at particular times in order to record such event information, e.g., via the questionnaire, within the compliance window according to the sampling schema of Table 7. An event scheduler 300 (FIG. 19) may be provided for this purpose and executed by the processor 102 of the meter 100, which an example thereof is discussed hereafter. In some embodiments, the user may enter event information based on event triggers. For example, when a strip is inserted into the meter, the meter may prompt the user to enter event information.

FIG. 19 depicts a process of the event scheduler 300. In step 302, a timer T synced with the clock 144 is incremented wherein in step 304, the processor 102 checks a structured sampling schema, such as included in a protocol file provided in memory 110 or 112, to see whether the current time T matches an alarm time for inputting event information. It is to be appreciated that the structured sampling schema provides daily times (and hence alarms) for such collections. Also, ΔT can be periodic or determined by another algorithm where the algorithm determines ΔT dynamically so as to meet Table 7 requirements. If so, then in step 306 the processor 102 provides an alarm, such as an audio signal via speaker 148, visual signal via display 108, tactile signal (e.g. vibrations), email message, SMS, etc. to the patient 142. In step 308, after the patient 142 acknowledges the alarm via use of the user interface 146, insertion of a strip 140 into the strip reader 138, or after some set period of time, such as via expiration of a count down timer, the processor prompts the patient 142 for entry of the event information, via the questionnaire displayed on the display 108 by the processor 102.

In step 310, if the processor 102 fails to detect an entry via the user interface 146, after expiration of another count down timer e.g., 300 seconds (or in other embodiments the timer can range from few minutes to half an hour, and preferably 5 to 10 minutes), then the processor 102 in step 312 resets the alarm for a future time T which is still within the compliancy window of Table 7 for collecting the event information. If an entry was made and detected in step 310, such as placed into temporary memory, such as main memory 110 via the processor accepting input from the user interface 146, then in step 314 the processor 102 stores the entry in secondary memory 112 in a manner discussed previously above.

If in step 304, the structured sampling schema in memory 110 or 112 does not have an alarm for the processor 102, then the processor 102 in step 316 will check for any triggered events, e.g. auto initiated via another running process of the meter 100 or patient initiated via the user interface 146. If none is detected, then the processor 102 loops back to step 302 and the processes of the event scheduler 300 repeat. If a trigger event is detected, then in step 318 the processor 102 checks whether an entry is needed for the triggered event, such as by doing a lookup in the profile file. If an entry is needed then the process goes to step 308, and if not, the process loops back to step 302 and repeats. It is to be appreciated that the scheduler 300 when executed by the processor 102 of the meter 100 indicates and consequently records a SMBG measurement and the associated event information (e.g., via running the questionnaire) in compliance with the measurement schema provided according to Table 7. Any unforeseen event is also enterable into memory 112 of the meter 100 at any time by the patient 142 via manually running the questionnaire on the meter 100 e.g. a triggered event in step 312. For example, non-prompted entry may occur in step 309 whenever the user decides to submit an entry that was not directly prompted by an alarm.

Returning to FIG. 18, in step 204, the processor 102 checks to determine whether the bG data was collected in a compliant manner. Data compliant in step 204 means that rules and guidelines to collect data which were either programmatically or manually complied to by the user. In one embodiment, compliance check includes: checking to see whether the number of days in the bG data meets the minimum number needed to satisfy the nDay requirement (i.e., a predetermined period, which in one embodiment is >2 weeks if an HCP desires to use a few weeks of data to predict a future HbAlC and true mean bG in order to revise a patient's current therapy or behavior in order to try achieve their targeted goals, >80 days for a result having a CV<3%, or any amount of days there between for a snapshot); and checking to see whether a minimum number of the samples (nSample) collected at a requested sampling event per the collection schema (i.e., a predetermined amount, which in one preferred embodiment is >45, but in other embodiments may some other amount that is reason for a patient's lifestyle as determined by the HCP) also satisfy the sampling time window requirement (e.g., WinSize<50 minutes). In one embodiment, the predetermined amount and period is read by the processor 102 from the structured sampling schema provided in memory. The result of the check on the collected bG data is either yes or no. In step 206, optionally, the processor 102 then checks for distribution of the time of bG samples with respect to the window center to see if there is a bias in the preferred time of post-prandial measurement. If there is, then in step 208 a correction for the bias may be added to the data. For example, if the time for bG measurement with respect to targeted post-prandial average time is biased then the algorithm will systematically alter the alert time in the future measurement of bG such as by alerting the patient later with respect to the targeted measurement time or may be raise an additional alert for measurement at a subsequent latter time and thus over period of days remove bias from the average time of measurement.

In another embodiment, an associated penalty in the precision of the estimated value may be flagged in step 208 such that a bias in time of bG measurement warning message is indicated with the provided results in step 214. After step 204, and optional step 206, if the bG data is compliant then the estimation processes Steps 210 and 212 are evoked. In step 210, the estimation process as mentioned previously above in reference to FIG. 7 bins together the data as per lifestyle as per event. The weighted mean bG is then determined by using the temporal weighting (harmonic weighting), whereby each of the weighted mean bG is then further time weighted by lifestyle related weight. The resulting value from step 210 is the estimate of the true mean bG value. Optionally, the estimated bG value is provided with an uncertainty window around the predicted value as is shown by FIGS. 16A-E. In step 212, the estimated HbAlC is then determined by solving the equation given in Table 7 using the estimated bG value from step 212, and the results then provided in step 214.

In another embodiment, an enhancement to the above model in Table 7 is to obtain an HbAlC value from an HbAlC assay, which can then be used as the patient specific intercept value c, instead of the given value of 0.5702. Such an embodiment is considered an estimated HbAlC with a one-point calibration. In still a further embodiment, another enhancement to the above model in Table 7, would be to obtain HbAlC using an HbAlC assay at two different points in time. These HbAlC values can then be used to determine a patient specific intercept value c and slope m. In such an embodiment, the two HbAlC values from the HbAlC assay not vary by more than +0.5% HbAlC to provide a good reliable slope m, assuming the assays are high quality (i.e., CV<2%). In another embodiment, the process 200 may then request whether the protocol file used for collection by the scheduler 300 needs updating in step 216. If so the protocol file is updated in step 218 via e.g., accepting user input via the user interface 146, e.g., from the processor 102 re-running a setup questionnaire on the display 108, receiving protocol changes from the HCP computer 134 when connected to the external device 132 such as, e.g., provided as a docking station, and combinations thereof. Afterwards, the process loops to step 202 and repeats. In another embodiment, Nathan's relation may be used for estimating HbAlc, wherein the estimated mean bG as described herein is used with Nathan's relation.

In still other embodiments, collection step 202, along with the scheduler 300, is solely performed on the meter 100, wherein process steps 204-218 are performed on the HCP computer 134. In such an embodiment, the HCP computer 134 may also provide additional capabilities, such as using the collected bG data with other models to perform comparisons with the model results according to the present disclosure. For example, in one embodiment, the HCP could run the collected bG data through a HbAlC population based model derived from continuously monitored glucose data.

As previously mentioned above, in one embodiment the alerting and collecting by the processing of bG measurements and associated context of the bG measurement at the daily times and the events can be specified by the structured sampling schema that is stored in memory. In one embodiment, the daily times specified by the structured sampling schema are post-prandial times. In another embodiment, the daily times specified by the structured sampling schema are three post-prandial times and another time. In still another embodiment, the events specified by the structured sampling schema is a specific time with respect to start of a meal. In yet another embodiment, one of the events specified by the structured sampling schema is an aspect of glucose behavior related to the estimated true mean bG value, which in one embodiment, the aspect is a bG mean to peak value. In still another embodiment, the daily times specified by the structured sampling schema are at about 140 to about 240 minutes after a meal time. In a further embodiment, the daily times and the events specified by a structured sampling schema are tailored to a daily lifestyle pattern of the patient. In yet another embodiment, the daily times specified by the structured sampling schema range from about 140 to about 240 minutes after a meal time in accordance with the daily lifestyle pattern of the patient. In even yet another embodiment, such as that which can be used with the Type 2 patient population, a seven point measurement per day may be performed for three days. Such sets of measurements can be taken at regular time periods such as between every two or six weeks (such as every four weeks).

In another embodiment, the processor 102 is further programmed to weigh a bG measurement if collection of the bG measurement was within a time interval from the daily times specified by the structured sampling schema, whereby in one embodiment the time interval is at most ±50 minutes. It is to be appreciated that the time interval also captures the information of whether the measurements, which are typically performed by the patient at random times around a recommended time, are falling within or outside the time interval. Such information may be used by the processor 102 to evaluate whether the patient's lifestyle has been captured appropriately as reflected in the structured sampling schema and/or whether the patient requires training such as, for example, if a threshold number of measurement within the time interval is not meet over a period of time. For example, in one embodiment, if such a threshold number of measurements is not achieved, the processor 102 provides a message on the display 108 indicating a collection problem and can provide a recommendation, such as ways to improve collection compliancy. In still another embodiment, the processor 102 is further programmed to determine the estimated true mean bG value and the estimated HbAlC value from the weighted measurements of the collected bG measurements if a predetermined amount of the bG measurements per each of the daily times and the events has been collected. In one preferred embodiment, the predetermined amount is at least 80 days, and in another embodiment at least 60 days. In still another embodiment, the processor is further programmed to determine the estimated true mean bG value and the estimated HbAlC value from the weighted measurements of the collected bG measurements if the predetermined amount of the bG measurements per each of the daily times and the events has been collected, and if the collection of the bG measurements occurred within a predetermined period of at least 2 weeks.

Displaying Grouped Estimated Biological Values or Grouped Predicted Biological Values

As discussed above, while specific examples are presented herein of weighting bG measurements to determine mean bG values to then determine HbAlC values, other biological measurements can similarly be incorporated to estimate a patient's estimated (current) or predicted (future) condition based on the weighting of current and previous biological measurements. As used herein, “biological measurements” includes any type of measurement that provides insight into the patient's health with respect to diabetes. For example, biological measurements include, but are not limited to, bG measurements, HbAlC measurements, fructosamine, lipids, triglycerides, insulin concentration, etc. Accordingly, one or more of these biological measurements can be measured, weighted and used to determine an estimated or predicted biological measurement (either the same type of biological measurement that was obtained, or a different type of biological measurement, but one that can be determined from the type of biological measurement that was obtained). Moreover, the biological measurements can be grouped by one or more variables, such that the estimated or predicted biological values can be determined for each group in order to compare the effect each variable has on the patient's health.

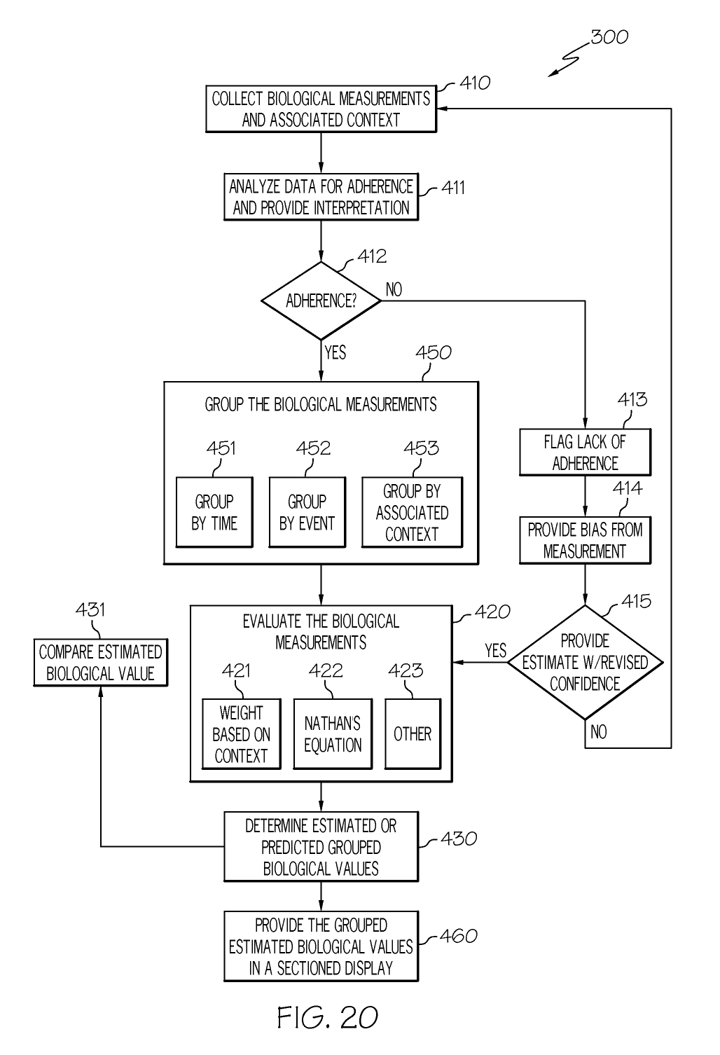

For example, referring now to FIG. 20, in some embodiments, the biological measurements collected comprise bG measurements, and the estimated/predicted values determined based on the weighted biological measurements comprise estimated HbAlC values. Therefore, the estimated HbAlC values determined from the weighted bG measurements as discussed above may be used in an informational delivery method 400 to provide grouped estimated HbAlC values in a sectioned display, i.e., a display comprising a plurality of sections. As used herein, “plurality of sections” refers to different areas on the display such that the estimated or predicted biological values for the different groups can be compared, as opposed to combined into a single value. For example, plurality of sections can include different sections of a pie graph, different adjacent bars on a bar graph, different plots on a graph, or any other format that allows for the comparison of estimated or predicted grouped biological values that are derived from the grouped biological measurements. Specifically, the estimated HbAlc values may be grouped by one or more variables (e.g., meal times, events, type of activity) so that a patient and/or physician may quickly assess the predicted impact different lifestyle components are contributing to the patient's health.

The informational delivery method 400 begins in step 410 by collecting biological measurements (e.g., bG measurements), and potentially other information such as associated context, in any manner discussed above. For example, the collection of bG measurements in step 410 can occur manually (e.g., using testing strips) or automatically (e.g., using a continuous bG meter) or simply comprise a transfer of previously obtained biological measurements, and potentially associated context, from a database (e.g., downloading the information from a bG meter to a physician's computer). In some embodiments, the collection of biological measurements may further be performed continuously or in accordance with a structured sampling schema wherein bG measurements were obtained at regulated times of the day such as within a prescribed period of time before and/or after meals, activities or other events. Such embodiments may help ensure the estimated or predicted biological values are obtained from a sufficient sample to reduce the effect of inconsistencies such as those that may occur when obtaining biological measurements at inconsistent times following meals or obtaining biological measurements arbitrarily throughout the day. In some embodiments, the structured sampling schema may comprise collecting measurements around a structured medication schema (i.e., collecting measurements as the patient takes a prescribed medication according to a structured medication schema). Furthermore, biological measurements may be collected in step 410 by a patient (e.g., self-testing), by a physician (e.g., laboratory testing) or by any third party.

Furthermore, the number of samples and the length of time in which the samples are obtained may be adjusted based on the patient. For example, in some embodiments, such as for patients having Type-2 diabetes, bG measurements may be obtained for 7-14 days before determining an estimated HbAlC value. In some embodiments, such as for patients having Type-1 diabetes, bG measurements may be obtained for 40 to 80 days. Other time period and measurement frequencies may alternatively be utilized to address the specific patients' lifestyle and pharmacological aspects or the specific problem being investigated.

In some embodiments, after or while the bG measurements (or other biological measurements) are collected in step 410, the data is analyzed for adherence in step 411 and an interpretation of the adherence is optionally provided. Specifically, the data collected in step 410 can be analyzed to determine whether the measurements were collected in a manner that complied with the testing protocol (e.g., whether measurements were taken at the proper times around an event within specified duration, whether measurements were taken for the proper number of days, whether the patient underwent any additional lifestyle changes that would influence the measurements, etc.). If the collected measurements were determined that they adhered to the testing protocol in step 412, then the bG measurements are evaluated in step 420.

If the measurements collected in step 410 are determined to not adhere in step 412 (such as missed measurements or measurements collected at incorrect times), then lack of adherence can be flagged in step 413. Specifically, the reason for lack of adherence can be presented to the patient, health care provider or any other relevant party so that any estimated values, if still determined, are reviewed with the lack of adherence in mind. In some exemplary embodiments, the bias from the measurements is provided in step 414 to the patient, health care provider, or other relevant party so that future measurements may be obtained to offset the bias from the measurements already collected. For example, if the patient routinely obtains measurements 30 minutes after they're supposed to, future measurements may be obtained 30 minutes before they were originally supposed to so that the bias of delayed measurements is counter balanced. Depending on the severity of the lack of adherence, the measurements may either still be used to determine estimated values with a revised level of confidence (i.e. accuracy) in step 415 and the collected data can be analyzed and interpreted.

Still referring to FIG. 20, the informational delivery method 400 further comprises grouping the biological measurements (e.g., bG measurements) in step 450 according to one or more set of variables. The one or more set of variables can comprise any time, event, associated context or other parameter that can be associated with the biological measurements such that grouping the biological measurements by the variables allows one to assess the impact of individual components on estimated or predicted biological values as will later be determined. For example, in some embodiments, the biological measurements may be grouped by time in step 451. Grouping by time can include grouping the biological measurements according to the time of the day in which the bG measurements were obtained. For example, the day may be broken down into a breakfast time interval (T_{BF}), a lunch interval (T_{LU}), a supper interval (T_{SU}), and a fasting interval (T_{FA}). Alternatively, grouping by time in step 451 can comprise grouping by certain weeks or parts of a week (e.g., weekends compared to weekdays), grouping by seasons (e.g., winter compared to fall), or grouping by any other relative time frame. These embodiments can be useful in comparing two time periods.

Still referring to FIG. 20, in some embodiments, grouping the biological measurements in step 450 comprises grouping the biological measurements by events in step 452. Grouping the biological measurements by event in step 452 can comprise grouping the biological measurements based on the occurrence of events associated with the bG measurements. Events can include the taking of a medication (e.g., whether any was taken, what type was taken, how much was taken), the performance of a physical activity (e.g., whether the patient exercises, what type of exercise was performed, how long did the patient exercise), the consumption of a particular type of food, or any other event that occurs in a patient's life which can be tracked (but still occurs with enough frequency so that variations on these events allow for the effective monitoring of their health). When events occur at the same time of the day, grouping by event 452 will essentially comprise grouping by time (i.e., the event and the time of the event are the same). However, when events occur at various times in a day, week, month or longer, grouping by the event will be distinct from grouping by regimented time frames. For example, where events consist of taking a medication, the biological measurements may be grouped based on the type of medication taken (or whether any medication was taken) and/or the amount of medication taken. Thus, one can group the biological measurements to appreciate the effect each event has on his or her estimated or predicted biological values to understand the relative impact (e.g., success) various medications, exercise regimens or other events have on his or her health.

In some embodiments, grouping the biological measurements in step 450 comprises grouping the biological measurements (e.g., bG measurements) by associated context in step 453. As discussed above, associated context can comprise variables on a patient's routine such as the size of a meal or the speed in which consumed food is digested. In such embodiments, not only are the biological measurements weighted by the associated context in step 420 to determine a more accurate estimated HbAlC value in step 430 (as will be discussed later herein), but the biological measurements collected in step 410 can be grouped by the same associated context such that the relative impact of variables within the associated context can be better appreciated.

The biological measurements may be grouped in step 450 automatically based on predetermined parameters (e.g., where a bG meter is programmed by default to group by time), or may be grouped based on the command of an operator. In some embodiments, the method may comprise prompting the operator to select the set of variables in which to group the biological measurements. For example, the operator may be prompted with multiple options such as time, events, associated context, or other variables which are available based on the known variables in which biological measurements were obtained. While specific examples have been provided of how biological measurements may be grouped in step 450 of informational delivery method 400, it should also be appreciated that the biological measurements may further be grouped by any other set of variables which may allow insight into their impact on the patient's estimated or predicted biological values.

Still referring to FIG. 20, the informational delivery method 400 further comprises evaluating the biological measurements (e.g., bG measurements) in step 420. Evaluating the biological measurements comprises interpreting the collected biological measurements using a selected protocol such that an estimated or predicted biological value can be determined in step 430. For example, in some embodiments, evaluating the biological measurements in step 420 comprises weighting the biological measurements based on associated context 421 as discussed above. In other embodiments, evaluating the biological measurements in step 420 comprises using Nathan's equation 422 with the collected measurements. In even other embodiments, evaluating the biological measurements in step 420 comprises using any other protocol in step 423 such that an estimated or predicted biological value can be determined in step 430. Evaluating the biological measurements in step 420 can be performed by any individual and/or machine, such as, for example, a computer, a bG meter and/or a personal digital assistant as discussed above. Once the biological measurements are evaluated, such as in accordance with one of the processes discussed above such that estimated true mean bG values can be determined, the estimated or predicted biological values (e.g., HbAlC values) are determined in step 430. The estimated/predicted biological values determined in step 430 may thus estimate or predict what the biological value (e.g., HbAlC level) will be for the patient as a result of his or her most recent biological measurement. For example, the patient and/or his or her physician may thus assess the relative impact of the estimated/predicted mean bG or HbAlc as a whole or as sectioned groups, and thereby appreciate the relative impact of the time, event and/or context associated with that bG measurement. Similar to evaluating the biological measurements in step 420, determining estimated or predicted biological values in step 430 may be performed by any individual and/or machine, such as, for example, a computer, a bG meter and/or a personal digital assistant.

In some embodiments, the estimated/predicted HbAlC values (or other estimated/predicted biological values) determined in step 430 are compared to the last measured actual HbAlC value (or other last measured biological value) in step 431. Comparing the estimated biological value with the actual biological value can provide the patient with the predicted increase or decrease in the HbAlC value as compared to their last actual measured HbAlC value. In these embodiments, the patient may then appreciate the effect their medication, lifestyle choices, etc. are having on their health. In some embodiments, the estimated biological value is compared to a target/reference biological value to appreciate the progress of the therapy. In some embodiments, the estimated/predicted biological value determined from one group of biological measurements is compared to the estimated/predicted value(s) determined from one or more other group(s) of biological measurements. In some embodiments, the estimated biological value determined for one time period can be compared to another biological value (either estimated or actual) from another time period. The reference values can be manually entered or can be retrieved from a storage device. For example, in some embodiments, the reference values are stored in a system such us a local system (e.g., the patient's bG monitor) or a remote system (e.g., a computer, server, or other storage device that can be accessed).

Still referring to FIG. 20, after the estimated or predicted biological values are determined in step 430, the grouped estimated HbAlC values are provided in a sectioned display in step 460. Specifically, the estimated or predicted biological values are provided such that each group is provided within at least one of a plurality of sections of the display. For example, where the biological values are grouped by time in step 451, the plurality of sections of the display will comprise sections for various time frames in which to display the respective group of estimated or predicted biological values. In some embodiments, the daily times in which bG measurements (or other biological measurements) were collected comprise a breakfast timeframe, a lunch timeframe, a supper timeframe and an overnight timeframe (e.g., a fasting timeframe). Similarly, the plurality of sections in the sectioned display could comprise a plurality of sections comprising a breakfast section, in which the grouped estimated or predicted biological values reflecting the impact of biological measurements taken during the breakfast timeframe are displayed, a lunch section, in which the grouped estimated or predicted biological values reflecting the impact of biological measurements taken during the lunch timeframe are displayed, a supper section, in which the grouped estimated or predicted biological values reflecting the impact of biological measurements taken during the supper timeframe are displayed, and an overnight section, in which the grouped estimated or predicted biological values reflecting the impact of biological measurements taken during the overnight timeframe are displayed.

Likewise, in embodiments where the estimated or predicted biological values are grouped by events in step 452, the plurality of sections in the sectioned display can, for example, comprise a first type of medication section, a second type of medication section, a no medication section, or the like. Additionally or alternatively, in some embodiments, the plurality of sections may comprise sections based on the amount or concentration of the medication. In even other embodiments, when the estimated or predicted biological values are grouped by other associated context variables in step 453, the plurality of sections of the sectioned display may be based off the different context variables (e.g., a large meal size section for estimated HbAlC values based on bG measurements taken after meals of a large size, a normal meal size section for estimated HbAlC values based on bG measurements taken after meals of a normal size, and a small meal size section for estimated HbAlC values based on bG measurements taken after meals of a small size). As such, the plurality of sections allows for the component-based display of grouped estimated or predicted biological values so that the effect of each component can be assessed. Optionally, an interpretation may be provided based on the grouped estimated or predicted values provided in the display conveying the relevant information such as the relative impact each component (i.e., variable) has on the patient, potential lifestyle changes, potential medication changes, etc.

Therefore, by grouping estimated or predicted biological values by events, one can quickly assess the relative success a prescribed therapy is having on the patient. For example, a patient, physician or other third-party can assess the relative impact attributed to the grouped estimated HbAlC values from each section of the sectioned display. Thus, when one section provides a greater impact to HbAlC values (such as when large meals account for the greatest impact on HbAlC values), its impact can quickly be visualized. This can be utilized to offer quick insight into the effectiveness of a therapy treatment (such as drug administration or exercise regimen) so that progress can be monitored. In addition, by allowing the quick assessment of the impact on the patient's HbAlC through the estimated HbAlC values, therapies can be quickly adjusted or discontinued (or lifestyles can be modified when possible) if they are not producing the expected or necessary results such as elevated glycemia or causing hypo glycemia. For example, in some embodiments, such as where the estimated HbAlC values are grouped based on events in which a new drug was and was not administered, the sectioned display can provide the effect on the patient's HbAlC when the drug was and was not administered. If administration of the drug produced little or no effect, the patient may adjust the drug amount, change the type of drug or stop the therapy to reduce unnecessary costs. The informational delivery method 400 allows this adjustment to occur in real time without waiting to collect actual HbAlC values for the patient during the next clinical visit.

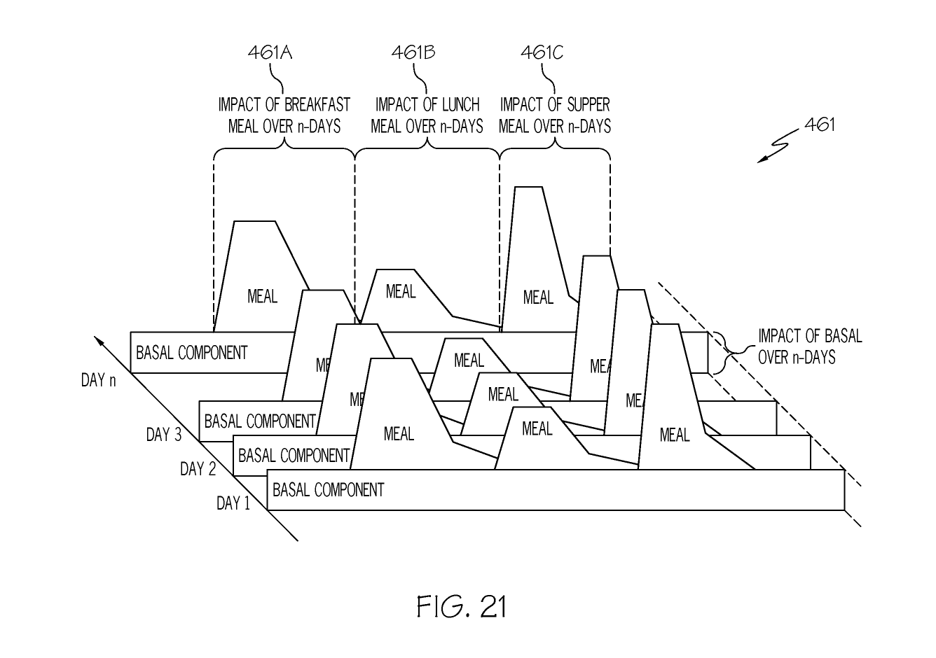

Referring now to FIGS. 20 and 21, an exemplary graphical visualization 461 is illustrated demonstrating the display of grouped biological measurements (such as HbAlC values) in accordance with step 450 of informational delivery method 400. Specifically, the exemplary visualization displays the biological measurements for each meal he or she has for multiple days grouped by the respective meal (i.e., breakfast, lunch and supper). Specifically, as illustrated in FIG. 21, the biological measurements are grouped into the first meal of each day 461A (i.e., breakfast) the second meal of each day 461B (i.e., lunch), and the third meal of each day 461C (i.e., supper). By grouping these relative impacts, the patient and health care provider can better understand the relative impact each component has to his or her breakdown of glucose excursion.

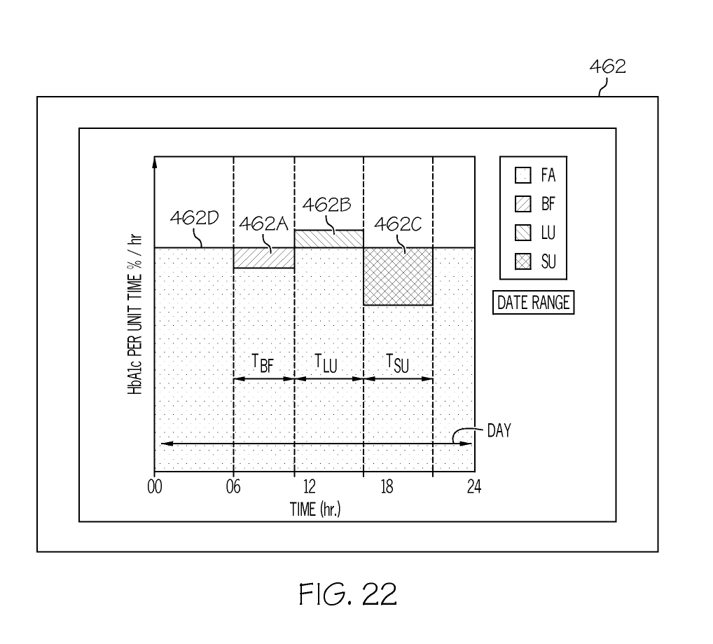

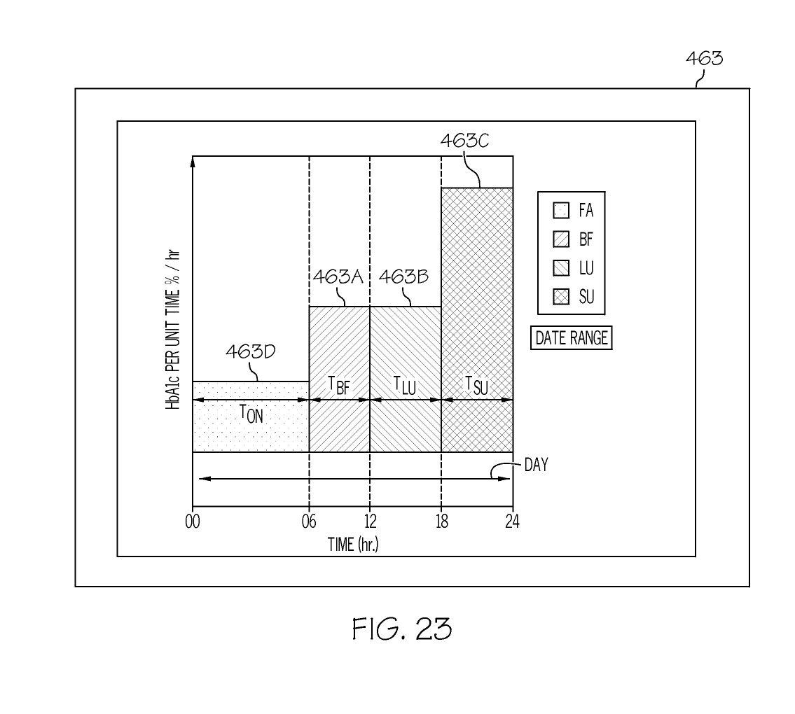

Referring now to FIGS. 20 and 22, a first exemplary sectioned display 462 is illustrated in which the contribution due to meal intake and insulin control action is displayed. In the first exemplary sectioned display 462, the bG measurements were grouped in step 450 according to the time of the day (i.e., a timeframe for breakfast T_{BF}, a timeframe for lunch T_{LU}, a timeframe for supper Tₛᵤ and a timeframe for fasting) and the estimated values were determined as described by Equation (29B) and Equation (30A) to show relative effect of meals with respect to a fasting state. In some embodiments, other comparative interpretations may alternatively or additionally be provided, such as providing absolute values (as illustrated in FIG. 23), median values, mode values, etc. The grouped estimated HbAlC values are then provided in step 460 in the sectioned display by comparing the contribution due to the meal intake as well as the insulin control action with respect to the basal (i.e., the fasting contribution). Specifically, the height and width of each section of the section display is generated as follows: the X-axis represents a 24 hr day, the width of the section is proportional to the duration of the section, the height is the value of HbAlc for the section divided by the width of the section. The area of the section represents the HbAlc value, the width of the section has units of hours and the height has the unit of HbAlC %/hour. As illustrated, the contribution from breakfast 462A and supper 462C is overly controlled with respect to the basal 462D, while the contribution of lunch 462B shows positive contribution to the Alc. By providing this first exemplary sectioned display in step 460 of informational delivery method 400, the patient, physician or any other party may quickly assess the relative impact of each meal using estimated HbAlC values to develop a revised therapeutic plan to address the relative contributions without waiting for new clinical HbAlC measurements.

Referring now to FIGS. 20 and 23, a second exemplary sectioned display 463 is illustrated in which each group is displayed in its own section 463A-D as an individual component independent of one another. Similar to the first exemplary sectioned display 462 of FIG. 22, the bG measurements were grouped in step 450 according to the time of the day (i.e., a timeframe for breakfast T_{BF}, a timeframe for lunch T_{LU}, a timeframe for supper T_{SU} and a timeframe for fasting) and the estimated HbAlC value for each group was determined so that the absolute values for each group are presented with respect to time so that the relative effect of each group is compared. The area of each section represents the respective contribution of HbAlc. The overall estimated HbAlc value can be displayed optionally along with the contribution by each of the section as HbAlc % value. Alternatively the values may be displayed in ratios or percentage of the overall HbAlc % value. The grouped estimated HbAlC values are then provided in step 460 in the sectioned display by displaying the contribution of each group independent of the others. As a result, the patient, physician or any other party can quickly assess the impact each section has (e.g., identifying that the supper section 463C contributes the greatest impact, as opposed to the breakfast section 463A or the lunch section 463B, on the basal 462D) and adjust the patient's therapy and/or lifestyle accordingly.

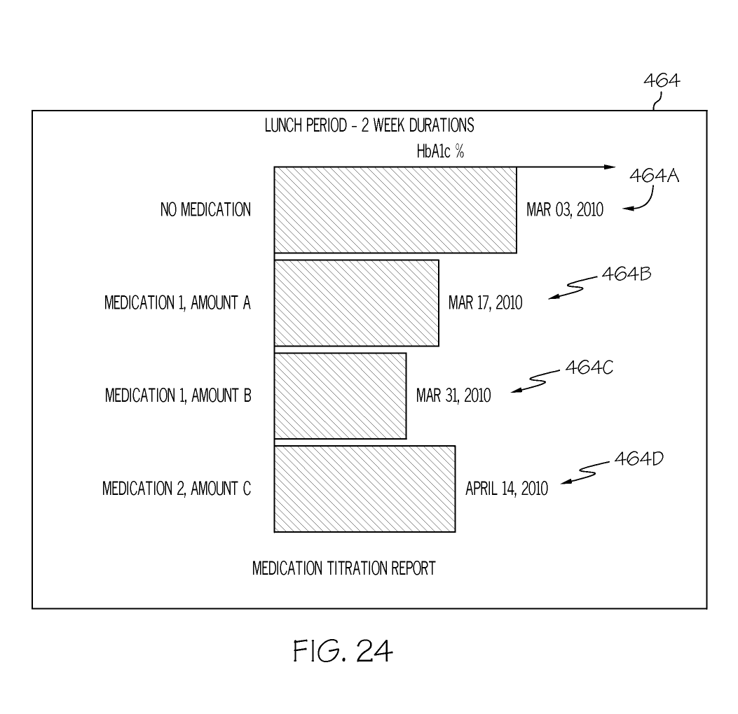

Referring now to FIGS. 20 and 24, a third exemplary sectioned display 464 is illustrated in which each section 464A-D is displayed to ascertain the relative impact of different types and amounts of medication. For the third exemplary sectioned display 464, the estimated/predicted HbAlC values were determined after the bG measurements were grouped in step 450 based on the time period in which a particular amount and type of medication was taken. Specifically, the estimated/predicted HbAlC values were grouped by a first two week time period in which no medication was taken 464A, a second two week time period (this 2 week period can be more or less) in which an “A” amount of “Medication 1” was taken 464B, a third time period in which a “B” amount of “Medication 2” was taken 464C, and a fourth time period in which a “C” amount of “Medication 3” was taken 464D. The estimated/predicted HbAlC values grouped in step 450 are then provided in step 460 in the sectioned display wherein a section corresponds to each time period (i.e., 464A, 464B, 464C, and 464D). As such, the relative impact of the patient's HbAlC can be assessed with respect to each group determine the relative success of each therapy (e.g., the effectiveness in reducing HbAlc/controlling glycemic excursion) of the different medication regimens.

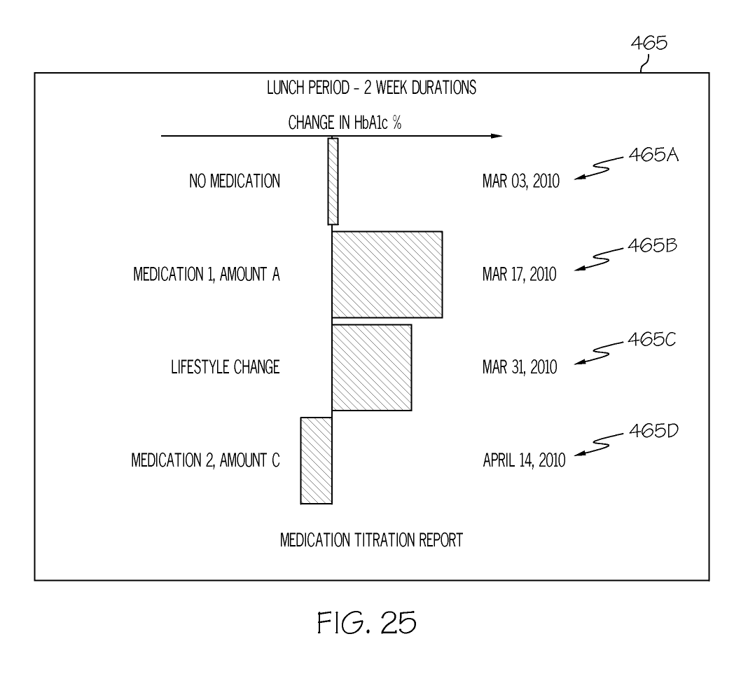

Referring now to FIGS. 20 and 25, a fourth exemplary sectioned display 465 is illustrated in which each section 465A-D is displayed to ascertain the relative impact of different types and amounts of medication and a lifestyle change (e.g., increased exercise or healthier eating). For the fourth exemplary sectioned display 465, the estimated/predicted change in HbAlC values were determined after the bG measurements were grouped in step 450 based on the time period in which a particular event was occurring, wherein the event consisted of taking a medication, taking no medication, or a lifestyle change. Specifically, the estimated/predict change in HbAlC values were grouped by a first two week time period in which no medication was taken 465A, a second two week time period in which an “A” amount of “Medication 1” was taken 465B, a third time period in which the patient underwent a lifestyle change 465C, and a fourth time period in which a “C” amount of “Medication 2” was taken 465D. The estimated/predict change in HbAlC values grouped in step 450 are then provided in step 460 in the sectioned display such that the impact of the second 465B, third 465C and fourth time frames 465D are illustrated compared to the impact of the first time frame 465A (i.e., when no medication was taken). As such, the patient, physician or any other party can quickly assess the impact of “Medication 1, Amount A,” “Lifestyle Change,” and “Medication 2, Amount C” compared to that of “No Medication.” The patient may then select or adjust a therapy based on the relative impact of each component. The change in HbAlc % is with reference to the case of no medication, or alternatively, to the selected reference type.

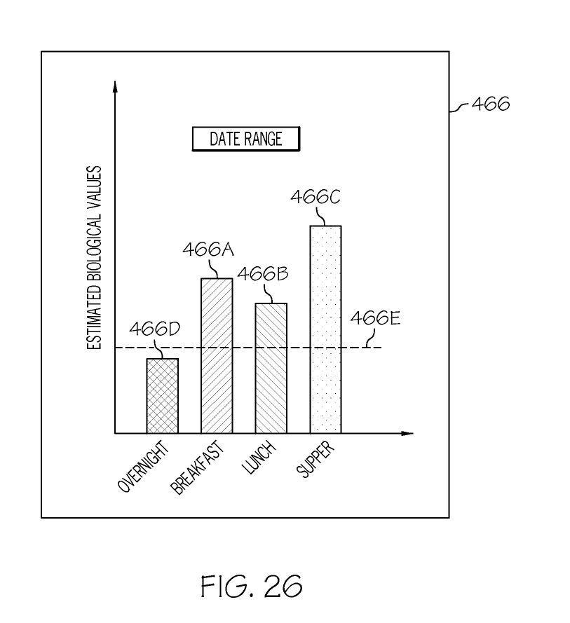

Referring now to FIGS. 20 and 26, a fifth exemplary sectioned display 466 is illustrated in which each section 466A-D is displayed to see how its current or previous estimated values (corresponding to the groups of breakfast 466A, lunch 466B, supper 466C and overnight (i.e., fasting) 466D), compares to the current or previous estimated values of other sections (which correlates with Equation (30) presented above in that the fasting component of Equation (30) is presented as the overnight section). For the fifth exemplary sectioned display 466, biological measurements were obtained (e.g., bG measurements) and weighted such that estimated biological values (e.g., predicted bG measurements or predicted HbAlC values) could be determined. Prior to determining the estimated biological values, the biological measurements were grouped into the four groups identified on the x-axis. Comparative values from each group (e.g., averages, absolute values, etc.) are thereby displayed in their respective sections so the relative impact of each group can be visualized. For example, the patient can determine the greatest impact is coming from the supper group and therefore they may be especially cognizant of the food they eat, associated medication and activities they undergo around supper. The y-axis may comprise different values depending on the type of estimated biological values. For example, where the estimated biological values comprise HbAlC, the y-axis may be defined in HbAlC %, mmol/mol or mg/dL. Furthermore, a reference target line 466E may also be displayed across the fifth exemplary sectioned display 466 corresponding to the target overall levels for the patient (or any other relevant target value or reference value) to ascertain where the estimated values project for the patient with respect to their targeted or previous measurements.

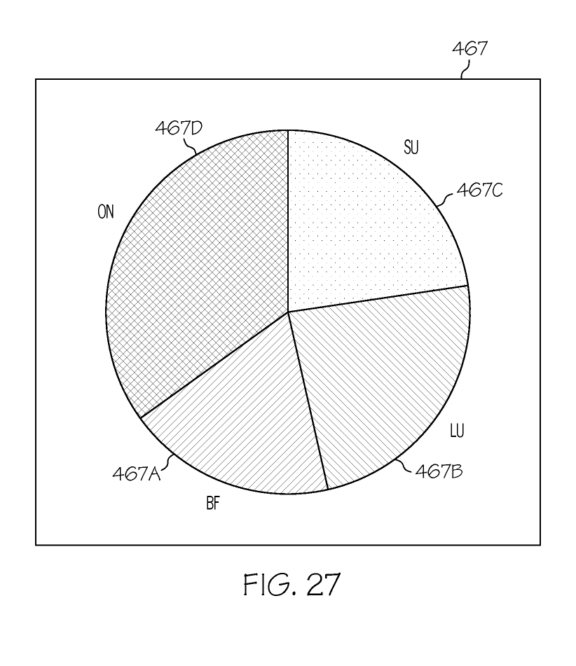

Referring now to FIGS. 20 and 27, a sixth exemplary sectioned display 467 is illustrated in which each section 467A-D is displayed in a pie chart to see how its estimated values (corresponding to the groups of breakfast 467A, lunch 467B, supper 467C and overnight (i.e., fasting) 467D), compares to the estimated values of other sections. For the sixth exemplary sectioned display 467, biological measurements were obtained (e.g., bG measurements) and weighted such that estimated biological values (e.g., predicted bG measurements or predicted HbAlC values) could be determined. Prior to determining the estimated biological values, the biological measurements were grouped into the four groups identified on the perimeter of the pie chart. Comparative values from each group (e.g., averages, absolute values, etc.) are thereby displayed in their respective sections so the relative impact of each group can be visualized.

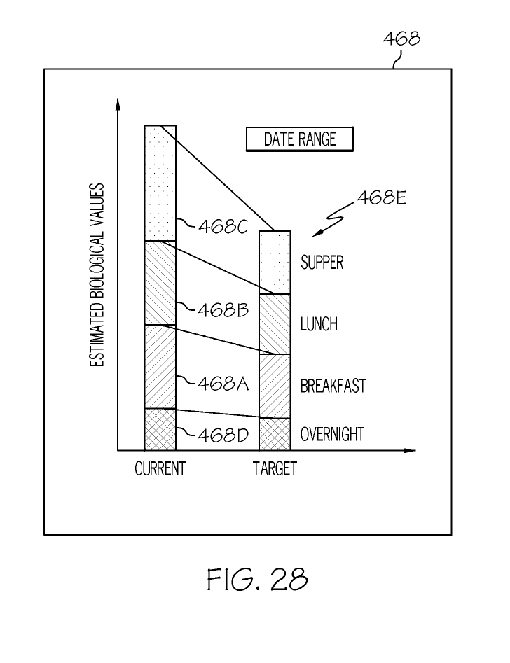

Referring now to FIGS. 20 and 28, a seventh exemplary sectioned display 468 is illustrated in which each section 468A-D is displayed to see how its estimated values (corresponding to the groups of breakfast 468A, lunch 468B, supper 468C and overnight (i.e., fasting) 468D), compares to the estimated values of other sections as well as target values. For the seventh exemplary sectioned display 468, biological measurements were obtained (e.g., bG measurements) and weighted such that estimated biological values (e.g., predicted bG measurements or predicted HbAlC values) could be determined. Prior to determining the estimated biological values, the biological measurements were grouped into the four groups identified along the bar chart. Comparative values from each group (e.g., averages, absolute values, etc.) are thereby displayed in their respective sections so the relative impact of each group can be visualized as an amount of the total biological value. The y-axis may comprise different values depending on the type of estimated/predicted biological values. For example, where the estimated/predicted biological values comprise HbAlC, the y-axis may be defined in HbAlC %, mmol/mol or mg/dL. Furthermore, a reference target bar 468E may also be displayed adjacent the estimated/predicted biological values corresponding to the target levels for each section to ascertain where the estimated/predicted values project for the patient with respect to their target values. Additional indicia such as “Hi”, “In Range”, or “Low” may also be displayed adjacent each section to indicate the patient's status compared to their target levels.

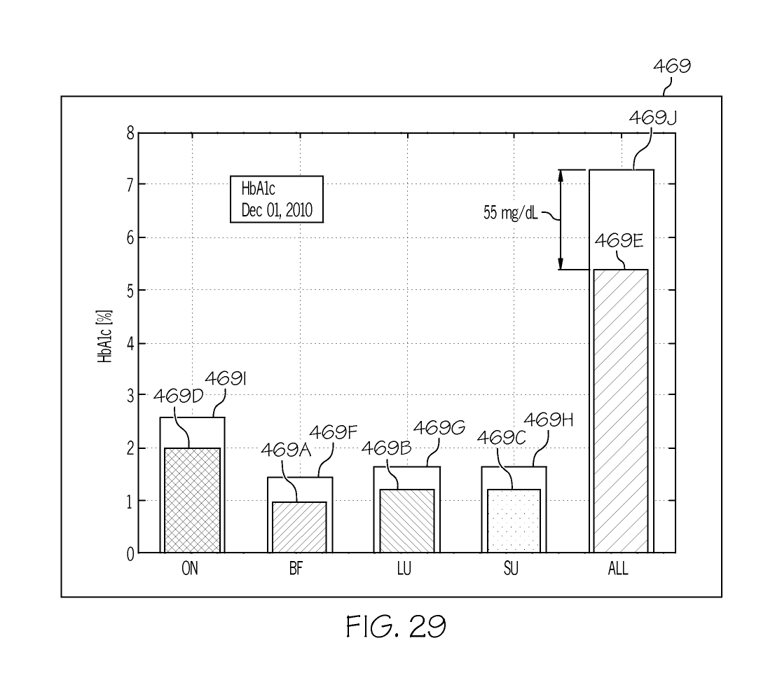

Referring now to FIGS. 20 and 29, an eighth exemplary sectioned display 469 is illustrated in which each section 469A-E is displayed to see how its estimated values (corresponding to the groups of breakfast 469A, lunch 469B, supper 469C, overnight (i.e., fasting) 469D, and all groups (i.e., total) 469E), compares to the estimated values of other sections as well as target values. For the eighth exemplary sectioned display 469, bG measurements were obtained and weighted such that estimated HbAlC values could be determined. Prior to determining the estimated biological values, the biological measurements were grouped into the groups identified along the bar chart. Comparative values from each group (e.g., averages, absolute values, etc.) are thereby displayed in their respective sections so the relative impact of each group can be visualized. Furthermore, reference target bars 469F-J may also be displayed adjacent the estimated biological values corresponding to the target levels for each section to ascertain where the estimated values project for the patient with respect to their target values.

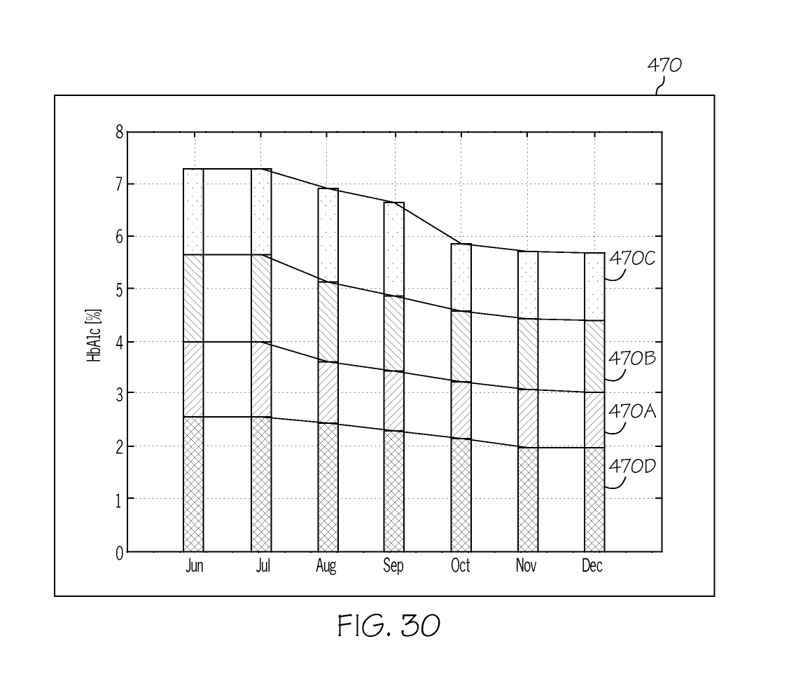

Referring now to FIGS. 20 and 30, a ninth exemplary sectioned display 470 is illustrated in which each section is displayed similar to seventh exemplary sectioned display 468 of FIG. 8, but wherein the biological measurements were grouped by month so that the patient's progress can be monitored. Specifically, the HbAlC % is illustrated for sections 470A-D (which can correspond to groups such as breakfast, lunch, supper and overnight (i.e., fasting)) and displayed in bar graph format for each month so that the patient's overall change by each month is visualized. Alternatively, the sections 470A-D may be grouped and displayed by weeks, seasons or any other temporal relationship.

While specific exemplary displays have been presented herein, it should be appreciated that other additional or alternative features may also be included. For example, such displays may selectively display additional information (such as the date range of measurements, labels/icons corresponding to the groups/events, etc), may be interactive (wherein the user can selectively change the data range, events, or other parameters that are displayed), may display actual values within each section of the sectioned display, may dynamically only display sections that are outside of target ranges, may be in color, gray scale or black and white, and/or contain any other relevant features for displaying grouped estimated biological values or grouped predicted biological values.

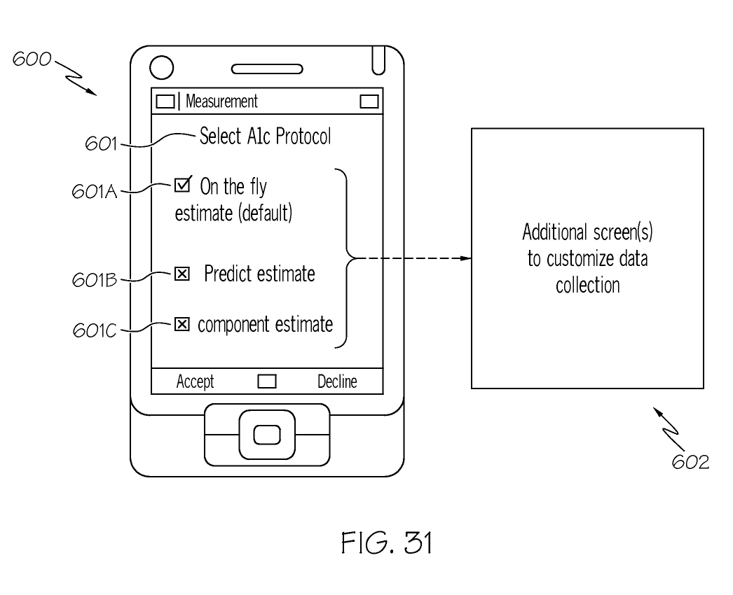

Referring now to FIG. 31, an exemplary textual screen 601 on an electronic device 600 (e.g., PDA, computer, etc.) is illustrated for prompting and conveying detailed information regarding the biological measurements and/or the estimated or predicted biological values presented in the exemplary displays discussed herein. Specifically, in some embodiments, after the user is presented their grouped estimated and/or predicted biological values in a sectioned display, they may be prompted or may request for details about various details such as, for example, the collected biological measurements, the method in which the estimated or predicted biological values were determined, how the estimated or predicted biological values were grouped, analysis constraints and/or the quality of results. For example, a user may desire to investigate the specific measurements that contributed to a display showing that their dinner-time estimated biological value is higher than desired. Therefore, the user may select that group (such as by selecting that section of the sectioned display, or following on-screen prompts to select that group) so that they may investigate the details of where that dinner-time measurements came from (i.e., what the measurements were, when they were recorded, what context was recorded relevant to those measurements, etc.).

In some embodiments, such as that illustrated in FIG. 31, the user may select or view what protocol is used to determine the estimated or prediction biological values (such as the HbAlC measurements as illustrated). For example, a plurality of protocols 601A, 601B, and 601C may be presented corresponding to different ways to determine estimated or predicted biological values (e.g., HbAlC values) based on biological measurements (e.g., bG measurements). In some embodiments, these selection screens can lead to additional selection screen 602 that allow for the further customization (or further analysis) of what is displayed. In other embodiments, the exemplary textual display 601 may be presented prior to displaying the sectioned display. In these embodiments, the exemplary textual display 601 may prompt the user regarding the collection of biological measurements (e.g., when to collect, how many were collected, how many more need to be collected, etc.), the protocol to determine the estimated or predicted biological values (e.g., weighting based on associated context, Nathan's relation, etc.), or the variables by which to group the estimated or predicted biological values (e.g., time, event, etc.).

Furthermore, while specific examples have been presented in grouping estimated biological values or predicted biological values (such as estimated HbAlC values according to step 450 of informational delivery method 400) and providing the grouped estimated biological values or grouped predicted biological values in a sectioned display (according to step 460 of informational delivery method 400), it should be appreciated that grouping may alternatively or additionally be performed in any other component-based methodology and be provided in any plurality of sections in the sectioned display that allows for the interpretation of the impact of each component.

Referring now to FIGS. 17 and 20, the informational delivery method 400 may be incorporated into a sectioned display device such as a bG meter 100 or similar electronic device comprising at least a display 108, an input terminal (such as a communications interface 124), memory (such as main memory or secondary memory 112) and a processor 102. In such embodiments, the input terminal (such as communication interface 124) collects both bG measurements and associated context of the bG measurements at daily times or events in accordance with step 410 of informational delivery method 400. The memory (such as main memory or secondary memory 112) stores the bG measurements, the associated context of the bG measurements and instructions. The processor 102 is in communication with the memory and operable to execute the instructions stored in the memory. Specifically, the instructions cause the processor to weight the bG measurements based on the associated context in accordance with step 420 of informational delivery method 400 and group the estimated HbAlC values based on a set of variables in accordance with step 450 of informational delivery method 400. The instructions further cause the processor to provide the grouped estimated HbAlC values in the display 108, such that the grouped estimated HbAlC values are each displayed within a plurality of sections.

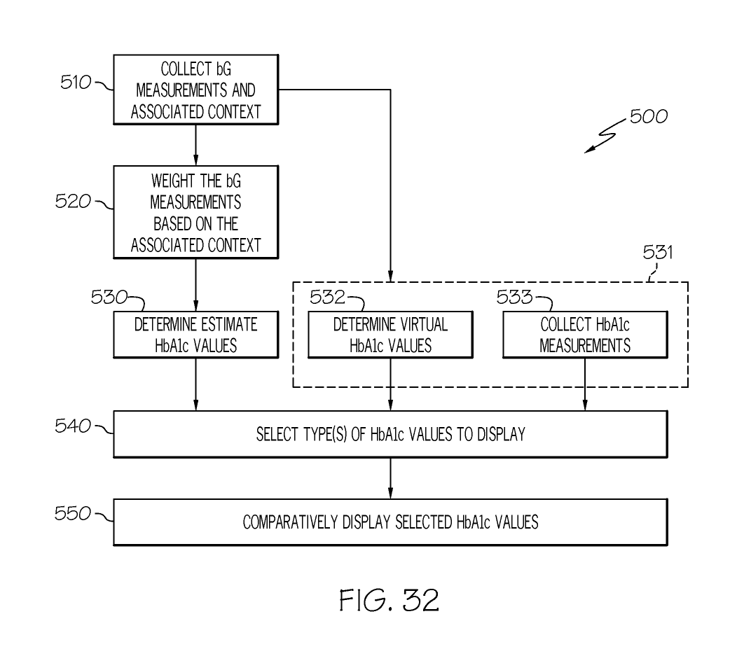

Referring now to FIG. 32, the estimated HbAlC values determined from mean bG values (or other estimated biological values determined from measured biological values) as discussed herein can be incorporated into a selective display method 500 in which one or more types of HbAlC values (e.g., actual, estimated, virtual) can be selectively displayed, such as in a comparative format. As illustrated in FIG. 32, the selective display method 500 comprises collecting bG measurements and associated context in step 510 (similar to collecting measurements and associated context in step 410 of informational delivery method 400 illustrated in FIG. 20). After bG measurements are collected in step 510, the bG measurements are weighted based on the associated context in step 520 of the selective display method 500 (similar to weighting the bG measurements on associated context in step 420 of informational delivery method 400 illustrated in FIG. 20). The selective display method 500 then comprises determining estimated HbAlC values in step 530 as well as additional types of HbAlC values in step 531. Specifically, estimated HbAlC values can be determined in step 530 using the weighted bG measurements as discussed herein.

Additional HbAlC values determined in step 531 can comprise any other HbAlC value actually measured from a patient or otherwise calculated from a patient based on other measurements such as bG measurements. For example, in some embodiments, an additional HbAlC value determined in step 531 can comprise a virtual HbAlC value determined in step 532. Virtual HbAlC values are those determined purely as a function of glucose concentration in which it as assumed that the glycation process is the same for each patient (and wherein bG values are not weighted based on the specific context of the patient). As such, while the patient specific physiological variability is not directly addressed in determining the value, glycemic control within patients can nonetheless be compared. Virtual HbAlC values may be determined in step 532 based on bG measurements collected in step 510. In some embodiments, an additional HbAlC value determined in step 531 can comprise the patient's actual HbAlC value as previously or currently measured in a clinical setting. In such embodiments, determining an additional type of HbAlc values may thereby simply comprise collecting actual HbAlC measurements in step 533 such that collecting the actual HbAlC measurements can comprise downloading a set of measurements or continuously updating a set of measurements as new values are determined.

After various HbAlC values are determined or collected through steps 530 and 531 of the selective display method 500, the types of HbAlC values to display are selected in step 540. Specifically, one can select any or all HbAlC values to display such that they can compare the different HbAlC values of the patient. For example, in some embodiments, the method may be incorporated in an electronic device such as a bG meter, PDA or computer. The operator may then be prompted to select which values to compare based on the different values determined/obtained in steps 530 and 531. For example, the operator may be able to select estimated HbAlC values (determined in step 530 which predicts the patient's HbAlC values based on previous bG measurements), actual HbAlC measurements (collected in step 533 which contains recent values actually measured from the patient) and/or virtual HbAlC values (determined in step 532 which estimates the patient's HbAlC value based on bG measurements, but relies on a population based model as opposed to weighting the individual bG measurements based on context). Furthermore, the user may also select the date range, events, or other parameter for which the HbAlC values are to be displayed.

After the types of HbAlC values are selected in step 540, the selected types of HbAlC values are displayed in step 550 of the selective display method 500. Specifically, the selected types of HbAlC values are displayed such that a user can see and/or compare the various HbAlC values. In some embodiments, the various values may be plotted on a common graph. For example, where estimated HbAlC values and actual HbAlC values are selected, then both sets of values may be plotted so a patient can visualize how his or her estimated HbAlC compares to his or her previously measured actual HbAlC results. In some embodiments, the value of the selected types of HbAlC values are displayed such that the patient can see how new values (such as estimated HbAlC values or virtual HbAlC values compare to previously measured actual HbAlC values, such as by including the percent change. Such embodiments may allow the patient to more quickly assess the effect of new therapeutic regimens and determine how such lifestyle changes are influencing his or her health or what actions they can take to improve upon current trends.

In summary, the embodiments of the present disclosure address the ability to provide grouped estimated biological values or grouped predicted biological values (such as estimated or predicted HbAlC values) in a sectioned display to patients, physicians and/or any other party so that the effect of new treatment regimens or other variations in a patient's life can be more quickly assessed without waiting for clinical measurements of actual values. For example, by weighting obtained bG measurements, estimated HbAlC values may be determined through calculating true mean bG values. The bG measurements can be grouped on a set of variables so that the estimated HbAlC values may be displayed so that the relative impact of different times, events or other context can be examined. The grouped HbAlC values may thus be delivered in a sectioned display to quickly asses the effect of each variable-based component towards the patient's HbAlC. Additionally, estimated HbAlC values may be determined along with other types of HbAlC values so that a user may select different types of HbAlC values for comparison. Such embodiments can allow for, among other things, a patient's previously measured actual HbAlC values to be compared with newly determined estimated HbAlC values to study the effect of recent therapeutic and/or lifestyle changes.

Having described the disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

What is claimed is:

1. A method for providing an estimated or predicted biological value in a sectioned display to assess a relative impact of a set of variables, the method comprising:
collecting biological measurements;
grouping the biological measurements based on the set of variables;
evaluating the biological measurements to determine grouped estimated biological values or grouped predicted biological values; and
providing the grouped estimated biological values or grouped predicted biological values within a plurality of sections in the sectioned display, wherein the plurality of sections correspond to the set of variables.

2. The method of claim 1, wherein the biological measurements comprise bG measurements.

3. The method of claim 1, wherein the estimated biological values comprise estimated or predicted glycated hemoglobin (HbAlC) values, fructosamine values, true mean blood glucose value, or triglyceride values.

4. The method of claim 2 wherein collecting bG measurements is performed via a bG meter.

5. The method of claim 1 wherein collecting biological measurements is performed in accordance to an event sampling schema.

6. The method of claim 1 wherein collecting biological measurements is performed in accordance to a structured sampling schema.

7. The method of claim 6 wherein the structured sampling schema comprises collecting biological measurements at post-prandial times, pre-prandial times or combinations thereof.

8. The method of claim 6 wherein the structured sample schema comprises collecting measurements around a structured medication schema.

9. The method of claim 6 further comprising verifying that collecting biological measurements adhered to the structured sampling schema.

10. The method of claim 1 wherein collecting biological measurements is performed after prompting triggered by an event.

11. The method of claim 10 wherein the event is taking a medication.

12. The method of claim 1 further comprising collecting associated context of the collected biological measurements.

13. The method of claim 12, wherein evaluating the biological measurements comprises weighting each of the collected biological measurements based on the associated context.

14. The method of claim 12, wherein the associated context comprises daily times or events.

15. The method of claim 1, wherein the set of variables comprises a timeframe in which the collected biological measurement was obtained.

16. The method of claim 15, wherein the timeframe comprises a breakfast timeframe, a lunch timeframe, a supper timeframe and an overnight timeframe.

17. The method of claim 16, wherein the plurality of sections comprises:
a breakfast section in which the grouped estimated biological values or grouped predicted biological values reflecting the impact of biological measurements collected during the breakfast timeframe are displayed;
a lunch section in which the grouped estimated biological values or grouped predicted biological values reflecting the impact of biological measurements collected during the lunch timeframe are displayed;
a supper section in which the grouped estimated biological values or grouped predicted biological values reflecting the impact of biological measurements collected during the supper timeframe are displayed; and
a fasting section in which the grouped estimated biological values or grouped predicted biological values reflecting the impact of biological measurements collected during the overnight timeframe are displayed.

18. The method of claim 1, wherein the set of variables comprises a type and/or amount of medication administered relative to the collected biological measurement.

19. The method of claim 18, wherein the plurality of sections comprises a first type of medication section and a second type of medication section.

20. The method of claim 18, wherein the plurality of sections comprises a first amount of a first type of medication section and a second amount of the first type of medication section.

21. The method of claim 18 further comprising evaluating an effect of the type and/or the amount of medication based on the grouped estimated biological values or grouped predicted biological values provided within the plurality of sections in the sectioned display.

22. The method of claim 1 further comprising providing an interpretation of the grouped estimated biological values or grouped predicted biological values provided in the sectioned display.

23. A sectioned display device for displaying grouped estimated biological values or grouped predicted biological values, comprising:
a sectioned display;
an input terminal for collecting biological measurements;
memory for storing the collected biological measurements and instructions; and
a processor in communication with the memory and operable to execute the instructions, the instructions causing the processor to group the biological measurements based on a set of variables, evaluate the biological measurements to determine grouped estimated biological values or grouped predicted biological values, and provide the grouped estimated biological values or grouped predicted biological values within a plurality of sections in the sectioned display, wherein the plurality of sections correspond to the set of variables.

24. The sectioned display device of claim 23, wherein the biological measurements comprise bG measurements.

25. The sectioned display device of claim 24, wherein the estimated or predicted biological values comprise estimated or predicted glycated hemoglobin (HbAlC) values.

26. The sectioned display device of claim 23, wherein the sectioned display device comprises a bG meter.

27. The sectioned display device of claim 26, wherein the bG meter further comprises an alarm that prompts an operator to collect biological samples according to a structured sampling schema stored on the memory.

28. The sectioned display device of claim 23, wherein the sectioned display device is in communication with an external server that evaluates the biological measurements to determine the estimated or predicted biological values.

29. The sectioned display device of claim 23, wherein the processor further verifies the collected biological measurements were collected in accordance with a structured sampling schema stored on the memory.

30. The sectioned display device of claim 23, wherein the instructions further cause the processor to provide an interpretation of the grouped estimated biological values or grouped predicted biological values provided in the sectioned display.

31. A method for selectively displaying a patient's glycated hemoglobin (HbAlC) based on various types of values, the method comprising:
collecting both bG measurements and associated context of the collected bG measurements at daily times or events;
weighting each of the collected bG measurements based on the associated context;
determining estimated or predicted HbAlC values from the weighted measurements of the collected bG measurements;
determining additional types of HbAlC values;
selecting which types of HbAlC values to display; and
displaying the selected types of HbAlC values.

32. The method of claim 31, wherein determining additional types of HbAlC values comprises determining virtual HbAlC values from the collected bG measurements.

33. The method of claim 32, wherein displaying the selected types of HbAlC values comprises displaying a difference between the estimated or predicted HbAlC values and the virtual HbAlC values.

34. The method of claim 31 wherein determining additional types of HbAlC values comprises collecting actual HbAlC values measured from a patient.

35. The method of claim 31, wherein displaying the selected types of HbAlC values comprises displaying a difference between the estimated or predicted HbAlC values and the actual HbAlC values.
